SEQUENCE LISTING (33 sequences)

SEQ ID NO: 1 - DNA, 2903 bp, Homo sapiens
  CDS (10)...(2076)
gattgtgagatgtgtcagaacttcttcattgacagctgtgctgcccatgggccccctacatttgtaaaggacagtgcagtggacaaggggcaccccaaccgttcagccctcagtctgcccccagggctgagaattgggccatcaggcatccctcaggctgggcttggagtatggaatgaggcatctgatctgccgctgggtctgcactttggcccttatgagggccgaattacagaagacgaagaggcagccaacaatggatactcctggctggtaagaagagcctgccatttcaccaaggggagaaactgctatgagtatgtggatggaaaagataaatcctgggccaactggatgaggtatgtgaactgtgcccgggatgatgaagagcagaacctggtggccttccagtaccacaggcagatcttctatagaacctgccgagtcattaggccaggctgtgaactgctggtctggtatggggatgaatacggccaggaactgggcatcaagtggggcagcaagtggaagaaagaggaaccaaagccagagatccatccatgtccctcatgctgtctggccttttcaagtcagaaatttctcagtcaacatgtagaacgcaatcactcctctcagaacttcccaggaccatctgcaagaaaactcctccaaccagagaatccctgcccaggggatcagaatcaggagcagcaatatccagatccacacagccgtaatgacaaaaccaaaggtcaagagatcaaagaaaggtccaaactcttgaataaaaggacatggcagagggagatttcaagggccttttctagcccacccaaaggacaaatggggagctgtagagtgggaaaaagaataatggaagaagagtccagaacaggccagaaagtgaatccagggaacacaggcaaattatttgtgggggtaggaatctcaagaattgcaaaagtcaagtatggagagtgtggacaaggtttcagtgttaaatcagatgttattacacaccaaaggacacatacaggggagaagctctacgtctgcagggagtgtgggcggggctttagctggaagtcacacctcctcattcaccagaggatacacacaggggagaagccctatgtctgcagggagtgtgggcggggctttagctggcagtcagtcctcctcactcaccagaggacacacacaggggagaagccctatgtctgcagggagtgtgggcggggctttagccggcagtcagtcctcctcactcaccagaggagacacacaggggagaagccctatgtctgcagggagtgtgggcggggctttagccggcagtcagtcctcctcactcaccagaggagacacacaggggagaagccctatgtctgcagggagtgtgggcggggctttagctggcagtcagtcctcctcactcaccagaggacacacacaggggagaagccctatgtctgcagggagtgtgggcggggctttagctggcagtcagtcctcctcactcaccagaggacacacacaggggagaagccctatgtctgcagggagtgtgggcggggctttagcaataagtcacacctcctcagacaccagaggacacacacaggggagaagccctatgtctgcagggagtgtgggcggggctttcgcgataagtcacacctcctcagacaccagaggacacacacaggggagaagccctatgtctgcagggagtgtgggcggggctttagagataagtcaaacctcctcagtcaccagaggacacacacaggggagaagccctatgtctgcagggagtgtgggcggggctttagcaataagtcacacctcctcagacaccagaggacacacacaggggagaagccctatgtctgcagggagtgtgggcggggctttcgcaataagtcacacctcctcagacaccagaggacacacacaggggagaagccctacgtctgcagggagtgtgggcggggctttagcgataggtcaagcctctgctatcaccagaggacacacacaggggagaagccctacgtctgcagggaggatgagtaagtcattagtaataaaacctcatctcaatagccacaaaaagacaaatgtggtcaccacacacttgcacaccccagctgtgaggtggcttcagcggaagtctgctgaccccttatattccccgagagtataaagagatcggaaataactgattaaacaaatccgccactttcatgactagagatgaggaagaacaagggatagttctgtaagtgttcgggggacatcagcatgtgtggttctttcccgcactgatcccctccattttttgtttgtttttttgcctcctgttctaataaattttgtctccatacaaatctgaaccccaagtgtgtacctcattcttcccttatcactgaaggcaagaagagtccagaagggccacagagaactcatgtgttcagctcaagactccacaggaattcaacccccagaaagacataaacttggagtccgtctggtttaattattggagaatcgattcccaagtccaggaagagaaatgtaagattctagaaagtcgcagcaggaaagggagttccctggtctcctgggaagtgtggcttcttctcctaatggacacctctcctctgctgccatactctcccttggctccccagtctcctctcctgatctcctccaatctctgtagcccaagatgtgaaagccagacaagaacacgcgtgtgtgtatatatgtgttcgggtgtgggggtatgtgccctccgtgtaggtaactgtgtgagtgtggggggtttcaagggtgtgttaggaacaacgctcaaaatcctaaggaaactgaacactcgaacgaaggattcttagcaa

SEQ ID NO: 2 - protein, 689 aa, Homo sapiens
MCQNFFIDSCAAHGPPTFVKDSAVDKGHPNRSALSLPPGLRIGPSGIPQAGLGVWNEASDLPLGLHFGPYEGRITEDEEAANNGYSWLVRRACHFTKGRNCYEYVDGKDKSWANWMRYVNCARDDEEQNLVAFQYHRQIFYRTCRVIRPGCELLVWYGDEYGQELGIKWGSKWKKEEPKPEIHPCPSCCLAFSSQKFLSQHVERNHSSQNFPGPSARKLLQPENPCPGDQNQEQQYPDPHSRNDKTKGQEIKERSKLLNKRTWQREISRAFSSPPKGQMGSCRVGKRIMEEESRTGQKVNPGNTGKLFVGVGISRIAKVKYGECGQGFSVKSDVITHQRTHTGEKLYVCRECGRGFSWKSHLLIHQRIHTGEKPYVCRECGRGFSWQSVLLTHQRTHTGEKPYVCRECGRGFSRQSVLLTHQRRHTGEKPYVCRECGRGFSRQSVLLTHQRRHTGEKPYVCRECGRGFSWQSVLLTHQRTHTGEKPYVCRECGRGFSWQSVLLTHQRTHTGEKPYVCRECGRGFSNKSHLLRHQRTHTGEKPYVCRECGRGFRDKSHLLRHQRTHTGEKPYVCRECGRGFRDKSNLLSHQRTHTGEKPYVCRECGRGFSNKSHLLRHQRTHTGEKPYVCRECGRGFRNKSHLLRHQRTHTGEKPYVCRECGRGFSDRSSLCYHQRTHTGEKPYVCREDE

SEQ ID NO: 3 - DNA, 6010 bp, Homo sapiens
  CDS (217)...(3399)
agccctcccaggtgtgggacagaaggggcagagggagggcagaggcttaaaggctgtgagcagtcagatcaaaaatgaagccagctcttcctcacagccggtagatggaagtcatttttccagggcctggaaggagttggtaggaagactagatttcagccagcccttgcgccctgtttgtgccagcaggcaccagcgaaccaaaagcaggtgtccatgtctgcttactctgtgccttcaacttttgcccaggcctcattgccagttcataaccaggtgctgccttccatcgagagtgtagatgggtccgaccctttggcaactctgcagacccctctaggcagactggaggccaaagaggaagaggatgaggatgaggacgaggacactgaggaagatgaggaagaagacggtgaggacacggatctggatgactgggagccagacccgccccggcccttcgacccacacgacttgtggtgtgaggagtgcaataacgcgcatgcttcagtgtgtccgaagcacggccccttgcacccgatccccaaccggccggtgctcacccgggccagggcgagcctccccctggtgctctacatagacaggtttctgggcggggtgttctccaagcggcgcatccccaagcgcacccagtttggccccgtggaggggcctctcgtcaggggctcggagctgaaagactgttacattcacctcaaggtttctcttgataaaggggacaggaaagaaagggatttacatgaagacctatggtttgagttgtctgatgagacgctttgtaactggatgatgtttgtacggccagcccagaatcacctggagcagaacctggtggcttaccagtatggccaccatgtgtattatacaaccataaaaaatgtggagcccaagcaggaactgaaggtgtggtatgccgcatcctatgctgagttcgtgaaccagaaaattcatgacatttctgaggaagaaaggaaagttcttcgagagcaagagaagaattggccctgctatgaatgtaaccgccgatttataagctcggagcagttgcaacagcatctcaattctcatgatgagaaactagatgtgtttagcagaacaagaggcagaggaaggggacgaggcaagaggcgattcggtccaggtcgacggccggggcgtcctccaaaatttatccgcctggaaatcaccagcgaaaatggggaaaagagtgacgatgggacacaggacttgctacattttcccacaaaggagcaatttgatgaggctgaaccagccactctgaatgggctggatcaaccagaacagaccactatcccaatccctcagctgccacaggaaacccagtcttccctggaacatgaaccagaaactcacaccctgcacctgcagccgcagcatgaagagagcgtggtgcccacccagagcacgctgacagccgacgacatgcgcagagccaagcgcatccgattggagctgcagaatgcagctcttcagcatctgtttattcggaagtccttccggccttttaaatgcttgcagtgtgggaaggccttccgggaaaaggacaaactggaccagcacttacgcttccatgggcgggaggggaactgcccactgacctgtgatctctgtaacaagggcttcatcagcagcacatccttggagagccacatgaagctccactcagaccagaagacttactcttgcattttttgcccagaatcctttgaccgccttgatttgttgaaagatcatgtggccattcatatcaatgatggctacttcacctgcccaacttgtaagaaacggttcccagattttatccaggtgaaaaaacacgtgcgcagcttccactcagaaaagatctaccagtgcacagagtgtgacaaggccttctgtcgccccgataaactgcgactccacatgctccggcattcggaccgcaaagacttcctgtgttccacctgtgggaagcaatttaagcgaaaagacaaactacgggaacacatgcagaggatgcataatcctgagagggaggccaagaaagccgaccgcatcagccgctccaagacgttcaagccccgcatcacgtccacagactacgacagcttcacgttcaagtgccgcctgtgcatgatgggcttccggcggcgcggcatgctggtaaatcacttatcgaagagacacccagacatgaagatagaagaggtgccagagttaactctacccatcataaaacccaatcgtgattacttttgtcagtattgcgataaggtttataaaagtgccagcaagcgcaaagcccacattctgaagaaccacccaggagcagagctcccaccgagcattcggaagctccgacccgctggtcctggagagccagaccccatgctgagcacacacacccagctgacgggcaccatcgccacccctcccgtctgctgtccccactgctccaagcagtacagcagcaagaccaagatggtccagcacattcgaaagaagcatccagagttcgcccagctctccaacaccatacacacaccactgacgacagctgtgatcagtgccaccccagcggttttgactacagacagcgccactggagagactgtggtgacgacggacctgctcacccaagcaatgacagaactgtcccagaccttaacgacagactaccgaacgccacaaggggattaccagagaattcagtacatccctgtgtcgcagtcggcgtctggcctccagcagcctcagcacatacagctgcaagtggttcaagtggcctcggccacttcccctcaccagtcacagcagtccactgtggatgttggccagctccatgatcctcagccctacccccagcacgccatccaggtgcagcacatccaggtatctgggcagccgttgagtccctcagcccagcaggctcagcaggggctcagcccctcccacatccagggcagttcttccacacaggggcaggctctgcagcagcagcagcagcagcagcagaattcctctgtgcagcacacgtacctgcccagtgcttggaattccttccgtggctattcatctgagattcaaatgatgacgcttcctccgggtcagtttgtgattacagacagtggtgtggcaactccagttactactggccaggtgaaggcggttacttcgggtcattatgtgttatcagaaagtcaatcagaattggaagaaaagcaaacttctgccctctctggtggagtccaggtcgagccacctgcacacagtgactccctggacccccagaccaacagccaacagcagaccacacagtacatcatcaccaccaccaccaacgggaacggaagcagcgaagtgcatatcaccaaaccatgacttccaccctggagcttgaatccagcacccacctcatgtctgttctcataagtctgaaagctctgacacgtagagctcttttgtaagatttattattcactcaagagttcggaaaccacagttttgtctctcatccatacactggggtttattttgccaaggtcatctttatcaaattgactcttcaaaccctcggtttttgccacagaacagagatctttcaggggaaagtagatttcgaggtggagagaatttgccttgctcttgagctggtcttttaacatactgacgagccttacttttcctttgtggaaatattaagtggcatattgtgttcataccaaaggtggagacaggatgtgccaagttcatggttactggacttcttattccaagccatggcaccaaaggagaaggggcattttgtgggtgtgcattaggtctgaactgctttatcttgttttagttttcagtaatttaacaaagggagcctgctgaataacaaaattaaaatgatgaaacaaaagtttgacagtgatattctgaagcaaaaactgtcatctaaatttttcttgctgattttttttaacttccctattttagaaaactattgttgggctgttggtagtgctttccacagatgctggtcctcagtggattaggtcagaagctcatgttctgatagttcttcattatttcacaaaggtggacaacctgaaagagaaaaggctgcttgatgtcagcagaatctaaacccctacattaaagctttgcctcccattctgtggttgtggtggtggtaagaaggagatattggtaccaagaaaaatttccaaaactattgagagagagagagagaaagtattgaaattttgttctgccttgcaggatccatttatcattcattggtaaaggatatattccttgttcttaccaagtgtacatttaactcttgctgacatgttcagtgtattttattacttttaatgctgtcttaaaatgaacattctttaaagttggacaggaaggtggcatttcttatttattaacatttttaactttaaacatattgtgactcatctagaccttttaataagacgatcaacttcacacttagggaaaaaattaaaatgtgatggccacttctaataattcaggtagttataagggatctggaagaaatccagtctttatgaatatactattagttgggcattaaaaaatcagtatatattttaactttttgtgacatgctaacacattttcagaataattattgcatagtagaatttattactccagaacagaaagaccttacttcatttcaaatgaagttaattaatttacatttctgggtaaaaactggttctataaattaagtagagtagcttctttatgataaagacaattttctgaaaagtgaacaattctgttaaaagaactttattgatgtgtagatttcgcactttatttctgagactgcgttcatgtagttgttccccaatgtttgagtactaagagaacttgagaaaattacttcataaacatatggaaactgaaaatggactctctttgtgacaaagatattcaacagaaaatattgaccctgcattgcaaaacacaggttcgggttccctaagtcccacagtgtaatgattacaaatatcctagtgtccggtctagaaagactttctaggagaagactagtggaattttctaaaggttcagttttagcttttataacttaaatttggccctgttacgttctttttaatttgaaccataaggtatctcccctcccttcaggaataacttattggaaaactgaaaagaatcactgaatggaatgttcattttatggcagttgttttaagttttaaaatacacagaggaaaatattgtggaaggacctctttgttgctttcccttctaagttgtcttcttcttcttcttcttcttcttcttctttggtccttaagtgaaataaagactctaaaactaatttgtatattatcagccagagatgcggatggcagtcgagccaaatcgcatggctttcagatcaggtattctgcacattcattccaaggtcatagatttttaaaaggacctggatttgaagagatggcaaatgatgagccatcagaaaacttaatttggaaaacatgtatgtagccagtgtggatattgtggcctctctcaagacacattgacactgtagacttcattcagtccagtgtgagtattttggagtaggttggatgtagattttgtttttatcattgatttgtaccgacagaaatagacatttcatcatgtaaaattcctgttattctggaaaaacctattgttttgatcttcttgttttctgacttggaagtatcctttcaaaaaaactcttaagatatctaggtctaaaaagcacttcatgagatgctaaagctgacccactggttgaaaatgttgaccctatcctgttatttaaatgtgaacatttattgtacattcagtgagttatagtgttaatagtcttgtgctatgcagcaggtgtaaaaattaataaatatattttttaataaacatgtttgtatgtgtgagtagtcatttc

SEQ ID NO: 4 - protein, 1061 aa, Homo sapiens
MSAYSVPSTFAQASLPVHNQVLPSIESVDGSDPLATLQTPLGRLEAKEEEDEDEDEDTEEDEEEDGEDTDLDDWEPDPPRPFDPHDLWCEECNNAHASVCPKHGPLHPIPNRPVLTRARASLPLVLYIDRFLGGVFSKRRIPKRTQFGPVEGPLVRGSELKDCYIHLKVSLDKGDRKERDLHEDLWFELSDETLCNWMMFVRPAQNHLEQNLVAYQYGHHVYYTTIKNVEPKQELKVWYAASYAEFVNQKIHDISEEERKVLREQEKNWPCYECNRRFISSEQLQQHLNSHDEKLDVFSRTRGRGRGRGKRRFGPGRRPGRPPKFIRLEITSENGEKSDDGTQDLLHFPTKEQFDEAEPATLNGLDQPEQTTIPIPQLPQETQSSLEHEPETHTLHLQPQHEESVVPTQSTLTADDMRRAKRIRLELQNAALQHLFIRKSFRPFKCLQCGKAFREKDKLDQHLRFHGREGNCPLTCDLCNKGFISSTSLESHMKLHSDQKTYSCIFCPESFDRLDLLKDHVAIHINDGYFTCPTCKKRFPDFIQVKKHVRSFHSEKIYQCTECDKAFCRPDKLRLHMLRHSDRKDFLCSTCGKQFKRKDKLREHMQRMHNPEREAKKADRISRSKTFKPRITSTDYDSFTFKCRLCMMGFRRRGMLVNHLSKRHPDMKIEEVPELTLPIIKPNRDYFCQYCDKVYKSASKRKAHILKNHPGAELPPSIRKLRPAGPGEPDPMLSTHTQLTGTIATPPVCCPHCSKQYSSKTKMVQHIRKKHPEFAQLSNTIHTPLTTAVISATPAVLTTDSATGETVVTTDLLTQAMTELSQTLTTDYRTPQGDYQRIQYIPVSQSASGLQQPQHIQLQVVQVASATSPHQSQQSTVDVGQLHDPQPYPQHAIQVQHIQVSGQPLSPSAQQAQQGLSPSHIQGSSSTQGQALQQQQQQQQNSSVQHTYLPSAWNSFRGYSSEIQMMTLPPGQFVITDSGVATPVTTGQVKAVTSGHYVLSESQSELEEKQTSALSGGVQVEPPAHSDSLDPQTNSQQQTTQYIITTTTNGNGSSEVHITKP

SEQ ID NO: 5 - DNA, 2208 bp, Homo sapiens
  CDS (250)...(1761)
gtcttgaggaccatctctcccggcagcataccgtgtggcttcacactgctctgcctctctgaacctcggtttcttcatctataaaatgggaataagagtaagccacctcaatggactgtgggaggcttaagtaaattgaagtgccatgcaagtagctagcatgcagttgcagctcaatgaatattatgatggccgcagatacgatggctacagctggggcacccatttcgggtcacaaggtagggttcaatgttgaagatggcagagccaattgcatccctgatgatcgtggagtgccgggcctgcctgagatgctcacctctcttcctttaccagagagagaaagacagaatgaccgagaacatgaaggagtgcttggcccagaccaatgcagccgtgggggatatggtgacggtggtgaatccgagccaggagtatggccagccctgctctaggagaccggactcctcggccatggaagttgagcccaagaaactgaaagggaagcgcgacctcatcgtgcccaaaagcttccagcaagtggacttctggttttgtgagtcctgccaggagtacttcgtggatgaatgcccaaaccatggccccccggtgtttgtgtctgacacaccggtgcccgtgggcatcccagaccgggcggcgctcaccatcccacagggcatggaggtggtcaaggacactagtggagagagtgacgtgcgatgtgtaaacgaggtcatccccaagggccacatcttcggcccctatgaggggcagatctccacccaggacaaatcagctggcttcttctcctggctgattgtggacaagaacaaccgctataagtccatagatggctcagacgagaccgaagccaactggatgaggtacgtggtcatctcccgggaggagagggagcagaacctgctggcgttccagcacagtgagcgcatctacttccgggcgtgcagggacatccggcctggggagtggctgcgggtctggtacagcgaggactacatgaagcgcctgcacagcatgtcccaggaaaccattcaccgcaacctggccagaggagagaagaggttgcagagggagaagtctgagcaggttctggataacccagaagacctgaggggtcccattcatctctctgtgctgagacagggcaaaagtccctacaagcgtggcttcgatgagggggatgtacacccccaagctaagaagaagaaaattgacctgattttcaaggatgttctggaggcctcactggaatctgcgaaggtggaagcccaccagttggccctgagcacctcactggtcatcaggaaagtccccaaataccaggatgacgcctacagtcagtgtgcaacaacaatgacccatggtgtgcagaatataggccagacccagggggagggggactggaaggtcccccagggggtctccaaggagccaggccaattggaggatgaagaagaggagccttcatcattcaaggccgacagtcctgccgaggcctcccttgcatctgaccctcatgaacttcccaccacctctttttgccctaactgtattcgcctaaagaagaaggttcgggagctccaggcagaattagacatgcttaagtctgggaaacttcctgagccccccgtattgccaccacaggtactggagctcccagagttctcggaccctgcaggtaagttggtttggatgagattattgtcggagggcagagtacgcagtgggctgtgtggagggtagcctaaagctctctgtggaaaccaccttccgggagacctgaggagtgtaacgtggaggcggctacctccgtgggtgggagcccaggtcctcagtgtctctggcagacccatcggcagctctgccaggtgctccatgtgttgcccttgtatcctccttgtcaataaaggaagttccgctgcagaaggggtgtgtgctgtgttcttgacccgttgcctttctctggtactggtgtcttaccccaaagcccaatttctaaacccagtctttctctgtccccagtctcaagcagggtgtcccactggagagatctcttggcttccctaacttagtccaggaacacagccttgttcttctcttcctgaatctctgtcctgccacacatggtcccagttccctagcctggagttctagaaggatggagagtgaggggatccaggccattca

SEQ ID NO: 6 - protein, 504 aa, Homo sapiens
MLKMAEPIASLMIVECRACLRCSPLFLYQREKDRMTENMKECLAQTNAAVGDMVTVVNPSQEYGQPCSRRPDSSAMEVEPKKLKGKRDLIVPKSFQQVDFWFCESCQEYFVDECPNHGPPVFVSDTPVPVGIPDRAALTIPQGMEVVKDTSGESDVRCVNEVIPKGHIFGPYEGQISTQDKSAGFFSWLIVDKNNRYKSIDGSDETEANWMRYVVISREEREQNLLAFQHSERIYFRACRDIRPGEWLRVWYSEDYMKRLHSMSQETIHRNLARGEKRLQREKSEQVLDNPEDLRGPIHLSVLRQGKSPYKRGFDEGDVHPQAKKKKIDLIFKDVLEASLESAKVEAHQLALSTSLVIRKVPKYQDDAYSQCATTMTHGVQNIGQTQGEGDWKVPQGVSKEPGQLEDEEEEPSSFKADSPAEASLASDPHELPTTSFCPNCIRLKKKVRELQAELDMLKSGKLPEPPVLPPQVLELPEFSDPAGKLVWMRLLSEGRVRSGLCGG

SEQ ID NO: 7 - DNA, 1437 bp, Homo sapiens
  CDS (61)...(1161)
cccgcccacctcccccgtcggcccggccgtcccccggcgccggggagctccgggccgcccatgatgggctccgtgctcccggctgaggccctggtgctcaagaccgggctgaaggcgccgggactggcgctggccgaggttatcacctccgacatcctgcacagcttcctgtacggccgctggcgcaacgtgctcggggagcagctcttcgaggacaagagccaccacgccagccccaagacagccttcaccgccgaggtgctggcgcagtccttctccggcgaagtgcagaagctgtccagcctggtgctgcctgcggaggtgatcatcgctcagagctccatccctggcgagggcctcggcatcttctccaagacgtggatcaaggcgggaaccgagatgggccccttcaccggccgcgtgatcgccccggagcacgtggacatctgcaagaacaacaacctcatgtgggaggtgttcaatgaggatggcacggtgcgctacttcatcgatgccagccaggaggaccaccggagctggatgacctacatcaagtgtgcacgtaacgaacaggagcagaacctggaggtggtccagatcggcaccagcatcttctacaaggccattgagatgatcccacctgaccaggaactgctggtgtggtacggaaactcacacaacaccttcctggggatcccaggtgtgcccgggctagaggaggaccagaaaaagaacaagcatgaggacttccacccggcggactcggcggctggccccgcgggccgcatgcgatgcgtcatctgccaccgcggcttcaactcgcgcagcaacctgcgctcgcacatgcgcatccacacgctggacaagcccttcgtgtgccgcttctgcaaccgccgcttcagccagtcgtccacgctgcgcaaccacgtgcgcctgcacacgggcgagcgcccctacaagtgccaggtgtgccagagcgcctactcgcagctggccggcctgcgcgcccaccagaagagcgcgcggcaccggccgcccagcaccgcgctgcaggcacactcgcccgcgctgcccgccccgcacgcgcacgcgcccgcgctcgccgccgccgccgccgccgccgccgccgccgccgcgcaccacctgccggccatggtgctgtgagcgcgcccgcgcccccgccgggccccgcgcgctcctgggtccccggcaccccggccccgcagcgcgactcgccctccagccccaacccccggcccggcgccgccgcggagccccgcgcgctggggttgcgccccggaggcggatctcaggcacccccgccttggcccgtgtcgcagatgaggacactgagggcggcgtccctcacccaggccacgcagctggtgcggctgttcggccgcctcctctgggagggggtccccctgcctggcctcg

SEQ ID NO: 8 - protein, 367 aa, Homo sapiens
MMGSVLPAEALVLKTGLKAPGLALAEVITSDILHSFLYGRWRNVLGEQLFEDKSHHASPKTAFTAEVLAQSFSGEVQKLSSLVLPAEVIIAQSSIPGEGLGIFSKTWIKAGTEMGPFTGRVIAPEHVDICKNNNLMWEVFNEDGTVRYFIDASQEDHRSWMTYIKCARNEQEQNLEVVQIGTSIFYKAIEMIPPDQELLVWYGNSHNTFLGIPGVPGLEEDQKKNKHEDFHPADSAAGPAGRMRCVICHRGFNSRSNLRSHMRIHTLDKPFVCRFCNRRFSQSSTLRNHVRLHTGERPYKCQVCQSAYSQLAGLRAHQKSARHRPPSTALQAHSPALPAPHAHAPALAAAAAAAAAAAAHHLPAMVL

SEQ ID NO: 9 - DNA, 3234 bp, Homo sapiens
  CDS (262)...(2412)
ccatgtgaccctctcgggtgggactctgcagctgcttcgcagcgcaactctctcaccaaactccgcgcccttgcgctagcggtgccaaaaggctcccgccccgattgaaaaggcgcagtgcatgcccgcccgcgtcactccgcgggcggaggacgcacgtcggggcgcggctctctggctagcgcgcagctccagctctgtcactcgcgcccttccaaggacctggagcacccgagcgcctgcctggtggcggcggcaacaatgcacggagccgccagagcgccagccaccagcgtgagtgccgactgctgcatcccggccggcttgcgcctcggaccggtgcctggtaccttcaagctgggcaagtacctgtcagaccgcagggagcccgggcctaagaaaaaggtattgaccattcagacctctgcccaccaggtgcgcatggtgagaggggagctggtggacgagtcggggggctcccctctggagtggatagggttaatccgggcagccagaaactcccaggaacagactctggaagctattgcagacttacccggaggacagatcttctaccgagcattgcgagacgtccagccaggggaggagctgacagtgtggtattctaactccttggctcagtggttcgacatccccaccacagcgactccgactcacgacgagaaaggggaggagcgctacatctgctggtactgctggaggacgtttagataccccaacagccttaaggcacacctgcgtttccactgcgtgttcagcggcggtggaggcggcgccttcctgcaccacgaacacgcggctcgccaaggcgccgtcccagcggctgatggcctcggtctctccccaaaacccccggcgcccgatttcgccgcgccttcccaggcaggaactttgcgaccccaccccctgggcccgccaccagttcaggcctgcggtgcgcgggagggcatcaagcgcgaggcctcttccgcgccctcggccacctcgccgaccccaggcaagtgggggcagcccaagaagggcaaggagcagctggaccgtgccctggacatgagcggagccgcccgaggacaagggcacttcctcggcatcgtgggcggctcctcggcgggggtcggcagcctggctttctaccccggcgtgcgctcagctttcaagcccgccggcctagcgagggcggcggcggccgctcacggcgacccctaccgggaggagagcagcagcaagcaaggagccggcctcgctttgggcaggctgctgggcgggggccgggcgtgcgggcgccccgggagcggggagaactcggcggcgggcggcgcgggtcaccaccatcaccaccacgcgcaccaccaccaccatcccaagtgcctgctcgctggggacccgccgccgccgccgccgcctggcctgccctgctctggggccctgcgcggcttccctctgctctccgtccccccggaagaggcgtccgccttcaagcacgtggagcgcgccccgcccgcagccgccgcgctgccaggagcgcgttatgcgcagctgccccctgcgccggggttgcccctcgagcgctgcgcgctgccgcccctcgacccgggcggtctcaaagcctatccgggtggtgagtgcagccacctgcccgccgtcatgccggcctttacagtctacaacggggagctgctctacggctcaccggccaccaccgcttattacccgctcaaattgcacttcggcgggctgctgaagtatccggagtccatctcctacttcagcgggcctgcagcggccgccctaagccccgccgagctggggtcgctggccagcatcgaccgagagatcgccatgcacaatcagcagctgtccgagatggctgccgggaagggtcgcggacgcctggactcggggacgttgccaccggccgtcgcggcggcgggaggcaccgggggcggcggcagcggaggcagcggcgcaggtaagcccaagaccggccacctgtgcctctactgtggcaagctgtactcgcgcaagtatgggctcaagatccacatgcggacgcacacgggctacaagccactcaagtgcaaagtctgtctgcggcccttcggcgaccccagcaatctcaacaagcacatccggctgcacgccgagggcaatacgccctaccgctgcgagttctgcggcaaggtacttgtgcgccgccgggacctggagcgacatgtcaagtcccgccaccctggccagagtctgctcgccaaagcgggcgacggcccgggtgccgagcccggctatcccccggagcctggggatcccaagagcgacgacagtgacgtggacgtctgcttcacagacgaccagagcgaccccgaggttgggggcggcggggagcgcgacttgtaacgagtcttcccgggaaggggcggggtgaggacagagaggagtcgagggtttattctcgcagtagaggaactcctggtggtgggaagagggacccaatggacaaaaccgtttttgtttttgagagggcgccagatttgaaacagtgagaggtcccacatctggtgctgaaactcagagcaacagttcagaggtggcgtaaatctggccacctggagagctcgagtgccaccagtacctccgcaccccgggcctctggacttcttggatgagctcaccctgaaccgcccaggcggtctgctcttggtgttcagaatcacatcaatgcgaacgtcacagcgccttcgagggcgcagattttaactgccacgtatttttaagttgtacttttctgtggaggaaattgtgccttttgaaacgacgttttgtgtgtgtatttcacgttagcatttcattgcataggcaaaacactagtcacaattgggtagatgtgacatccatatacttgtttacattttatctgttctcatgtcaaagactactccttgccccattgaatatatagtggtagcaggtgtacaaattggtcaagttgcaattatttatgagagaataatgataaatgtaaaatatctaaagcatgaatctaagagcacgcaatatataattttaaagaaaatattctatttggtagaatacaaatgtggtgtgtgttgttttataatgactgctgtacagtgggtatagtattttggttttggttccagattgtgcaatctttaagaaaaataaagatacaaacgagagttttgttcacttagt

SEQ ID NO: 10 - protein, 717 aa, Homo sapiens
MHGAARAPATSVSADCCIPAGLRLGPVPGTFKLGKYLSDRREPGPKKKVLTIQTSAHQVRMVRGELVDESGGSPLEWIGLIRAARNSQEQTLEAIADLPGGQIFYRALRDVQPGEELTVWYSNSLAQWFDIPTTATPTHDEKGEERYICWYCWRTFRYPNSLKAHLRFHCVFSGGGGGAFLHHEHAARQGAVPAADGLGLSPKPPAPDFAAPSQAGTLRPHPLGPPPVQACGAREGIKREASSAPSATSPTPGKWGQPKKGKEQLDRALDMSGAARGQGHFLGIVGGSSAGVGSLAFYPGVRSAFKPAGLARAAAAAHGDPYREESSSKQGAGLALGRLLGGGRACGRPGSGENSAAGGAGHHHHHHAHHHHHPKCLLAGDPPPPPPPGLPCSGALRGFPLLSVPPEEASAFKHVERAPPAAAALPGARYAQLPPAPGLPLERCALPPLDPGGLKAYPGGECSHLPAVMPAFTVYNGELLYGSPATTAYYPLKLHFGGLLKYPESISYFSGPAAAALSPAELGSLASIDREIAMHNQQLSEMAAGKGRGRLDSGTLPPAVAAAGGTGGGGSGGSGAGKPKTGHLCLYCGKLYSRKYGLKIHMRTHTGYKPLKCKVCLRPFGDPSNLNKHIRLHAEGNTPYRCEFCGKVLVRRRDLERHVKSRHPGQSLLAKAGDGPGAEPGYPPEPGDPKSDDSDVDVCFTDDQSDPEVGGGGERDL

SEQ ID NO: 11 - DNA, 2210 bp, Homo sapiens
  CDS (203)...(1915)
aattccctaccctcgacctgtcgatgccccgcggccccgcccgccctcttaagcctggctcagccctcagggcccgcccgaagtctaccgagcccgagtggcctaccgagcccgagtggccccgcagcgtccaggaggcgcccgctccgcggtggcgctcttggaggtggtgtcggagagccgccgagcgtgcggtcccgggatggctctaccccggccaagtgaggccgtgcctcaggacaaggtgtgctacccgccggagagcagcccgcagaacctggccgcgtactacacgcctttcccgtcctatggacactacagaaacagcctggccaccgtggaggaagacttccaacctttccggcagctggaggccgcagcgtctgctgcccccgccatgccccccttccccttccggatggcgcctcccttgctgagcccgggtctgggcctacagagggagcctctctacgatctgccctggtacagcaagctgccaccgtggtacccaattccccacgtccccagggaagtgccgcccttcctgagcagcagccacgagtacgcgggtgccagcagtgaagatctgggccaccaaatcattggtggcgacaacgagagtggcccgtgttgtggacctgacactttaattccaccgccccctgcggatgcttctctgttacctgaggggctgaggacctcccagttattaccttgctcacccagcaagcagtcagaggatggtcccaaaccctccaaccaagaagggaagtcccctgctcggttccagttcacggaggaggacctgcacttcgttctgtacggggtcactcccagcctggagcacccagccagcctgcaccatgcgatttcaggcctcctggtccccccagacagctctggatctgattctcttcctcaaactctggataaagactcccttcaacttccagaaggtctatgcctcatgcagacggtgtttggtgaagtcccacattttggtgtgttctgcagtagttttatcgccaaaggagtcaggtttgggccctttcaaggtaaagtggtcaatgccagtgaagtgaagacctacggagacaattctgtgatgtgggagatctttgaagatggtcatttgagccactttatagatggaaaaggaggtacggggaactggatgtcctatgtcaactgtgcccgcttccccaaggagcagaacctagttgctgtgcagtgtcaagggcatatattttatgagagctgcaaagagatccatcagaaccaagagctccttgtgtggtatggagactgctatgagaaatttctggatattcctgtgagccttcaggtcacagagccggggaagcagccatctgggccctctgaagagtctgcagaaggctacagatgtgaaagatgtgggaaggtatttacctacaaatattacagagataagcacctcaagtacaccccctgtgtggacaagggcgataggaaatttccctgttctctctgcaaacgatcctttgagaagcgggaccggcttcggatccacattcttcatgttcatgagaagcaccggcctcacaagtgttctacatgtgggaaatgtttctctcagtcttccagcctaaacaaacacatgcgagtccactctggagacagaccataccagtgtgtgtattgtactaagaggttcacagcctccagcatactccgcacacacatcaggcagcactccggggagaagcccttcaaatgcaagtactgtggtaaatcttttgcatcccatgctgcccatgacagccatgtccggcgttcacacaaggaggatgatggctgctcatgcagcatctgtgggaaaatcttctcagatcaagaaacattctactcccacatgaagtttcatgaagactactagccctgccaggcacaatgactcacgcctgtaatcccagcactttgggaggcagaggtgggtggatcactcaagtccaggagttcgagaccagcctgggcaacatggtgaaatcctgtctctaccaaaaaaatacaaaaatcagctgggggtggtggcacatgcctgtggttccagccactcaggaggtcgaggtggcgggatggtttgagcacaggagacggaggttgctgtgagctgagatcgccccactgcttttcaacctgggtgacagaaccagaccctgtctcaaaac

SEQ ID NO: 12 - protein, 571 aa, Homo sapiens
MALPRPSEAVPQDKVCYPPESSPQNLAAYYTPFPSYGHYRNSLATVEEDFQPFRQLEAAASAAPAMPPFPFRMAPPLLSPGLGLQREPLYDLPWYSKLPPWYPIPHVPREVPPFLSSSHEYAGASSEDLGHQIIGGDNESGPCCGPDTLIPPPPADASLLPEGLRTSQLLPCSPSKQSEDGPKPSNQEGKSPARFQFTEEDLHFVLYGVTPSLEHPASLHHAISGLLVPPDSSGSDSLPQTLDKDSLQLPEGLCLMQTVFGEVPHFGVFCSSFIAKGVRFGPFQGKVVNASEVKTYGDNSVMWEIFEDGHLSHFIDGKGGTGNWMSYVNCARFPKEQNLVAVQCQGHIFYESCKEIHQNQELLVWYGDCYEKFLDIPVSLQVTEPGKQPSGPSEESAEGYRCERCGKVFTYKYYRDKHLKYTPCVDKGDRKFPCSLCKRSFEKRDRLRIHILHVHEKHRPHKCSTCGKCFSQSSSLNKHMRVHSGDRPYQCVYCTKRFTASSILRTHIRQHSGEKPFKCKYCGKSFASHAAHDSHVRRSHKEDDGCSCSICGKIFSDQETFYSHMKFHEDY

SEQ ID NO: 13 - DNA, 2965 bp, Homo sapiens
  CDS (112)...(2964)
cgctgccccgtccacgccgcccgcgccccgcagtcccacccgcaggacccccggccgcgccagggtctcgcctgcgccccccgcgcccgcccgcggactacaagtcccgccatgccccgccgccgccccccggcttccggtgctgcgcagtttccggagcggatcgcaacccggagtccggatccgatcccgctctgcacattccaaaggcaaccgcgcgccgccccggtccagccgccatgccgactgttctttgttacattcgccggctgcgggcaccgttggcgatcggagtcaaaacccggctggatttcccggagccgctccgggatcgccctgcgcgccgcccgcccgccggggtcttcgccgccccggcccgcggccccgcggcccccgccgccgggcggcgtcgtcgccgaggccccaggggatgtcgttatcccccgccctcgggtacagcccatgcgggtcgcacgggggggtccctggacccccaaccccgcgtttagagaagctgagtcctggtcccagattgggaaccagagggtcagtgagcagctcttggaaacatctctagggaatgaggtgtccgacactgagccactgagccctgcgagtgcaggcctgcgacgcaatcctgccctacctcctggaccctttgcacaaaacttttcctgggggaaccaggaaaatctgcccccagccctggggaagattgcgaacggaggaggaactggggcaggcaaggccgaatgcggctatgaaactgagtcacacttgctagagccgcacgagatacctttgaacgtgaatacacacaagttcagtgactgtgagtttccatatgagttttgcacggtctgcttttcacccttcaagctgctggggatgagcggggtggagggcgtgtggaatcagcattcaagaagtgccagcatgcacactttcctaaaccactcagcaacgggcatcagagaagcaggttgcaggaaggacatgcccgtgtcagagatggctgaagatgggagcgaagagatcatgttcatctggtgtgaagactgcagccagtaccacgactccgaatgtcccgagctgggcccagtggtcatggtcaaagactcctttgtgttaagcagggcaaggtcttggcctgccagcggacacgtgcacacccaggcggggcaggggatgcggggttatgaggacagggacagggctgacccacagcagcttccagaagcagtccctgcaggcctggtgaggcggctcagtgggcagcagctgccctgccgttccaccctcacctgggggaggctgtgccacctggtggcccagggcaggtcatcccttcctcccaacttggagatcagacgactggaagatggagccgagggggtgttcgccatcactcagctcgtcaagcggacacagttcggtccctttgagtccaggagggtcgccaaatgggaaaaggagtctgcatttcccctgaaggtgttccagaaggacgggcaccccgtgtgcttcgacacctccaacgaggatgactgcaactggatgatgctggtgcggccagcggcggaggccgagcaccagaacctgacggcctaccagcacggcagcgacgtgtacttcaccacctccagagacatccccccgggtaccgagctgcgcgtgtggtatgcggccttctatgccaagaagatggacaagcccatgctgaagcaggccggctctggcgtccacgctgcaggcaccccagaaaacagcgcccccgtggagtcggagcccagccagtgggcgtgtaaagtgtgttctgccaccttcctggagctgcagctcctcaatgaacatctgttgggccacttagaacaagccaaaagccttcctccaggcagccaaagcgaggcagcagctcccgagaaggagcaggacacaccccggggggaaccccctgcagtgcccgagagcgagaatgttgccaccaaagaacagaagaaaaagcctcgaagggggagaaaacccaaagtgtccaaagctgagcagcctctagtcatcgtggaagacaaggaacccacagagcaagtggcagagatcattaccgaggtccctccggatgagcctgtgagtgcaacgccagatgagcggatcatggagctggttctggggaagctggccaccaccaccactgacaccagctcggttccaaagttcacccatcatcagaataacaccatcacgctcaagaggagcttaattctctcaagcagacacggcatccggcgcaagctcatcaaacagctcggggagcacaagcgggtttaccagtgcaatatctgcagcaagatcttccagaacagcagcaacctgagcaggcacgtgcgctcgcatggtgacaagctgtttaagtgcgaagagtgtgcaaaattgttcagccgcaaagagagcctaaagcagcacgtttcctacaagcacagcaggaacgaggtggacggcgagtacaggtaccgctgcggcacttgtgagaagaccttccgcatcgagagcgcgctggagttccacaactgcaggacagatgacaagacgttccaatgtgagatgtgtttcagattcttctccaccaacagcaacctctccaagcacaagaagaagcacggcgacaagaagtttgcctgtgaggtctgcagcaagatgttctaccgcaaggacgtcatgctggaccaccagcgccggcacctggaaggagtgcggcgagtgaagcgagaggacctggaggccggtggggagaacctggtccgttacaagaaggagccttccgggtgcccggtgtgtggcaaggtgttctcctgccggagcaatatgaacaagcacctgctcacccacggggacaagaagtacacctgcgagatctgcgggcgcaagttcttccgcgtggatgtgctcagggaccacatccatgtccact

SEQ ID NO: 14 - protein, 951 aa, Homo sapiens
MPRRRPPASGAAQFPERIATRSPDPIPLCTFQRQPRAAPVQPPCRLFFVTFAGCGHRWRSESKPGWISRSRSGIALRAARPPGSSPPRPAAPRPPPPGGVVAEAPGDVVIPRPRVQPMRVARGGPWTPNPAFREAESWSQIGNQRVSEQLLETSLGNEVSDTEPLSPASAGLRRNPALPPGPFAQNFSWGNQENLPPALGKIANGGGTGAGKAECGYETESHLLEPHEIPLNVNTHKFSDCEFPYEFCTVCFSPFKLLGMSGVEGVWNQHSRSASMHTFLNHSATGIREAGCRKDMPVSEMAEDGSEEIMFIWCEDCSQYHDSECPELGPVVMVKDSFVLSRARSWPASGHVHTQAGQGMRGYEDRDRADPQQLPEAVPAGLVRRLSGQQLPCRSTLTWGRLCHLVAQGRSSLPPNLEIRRLEDGAEGVFAITQLVKRTQFGPFESRRVAKWEKESAFPLKVFQKDGHPVCFDTSNEDDCNWMMLVRPAAEAEHQNLTAYQHGSDVYFTTSRDIPPGTELRVWYAAFYAKKMDKPMLKQAGSGVHAAGTPENSAPVESEPSQWACKVCSATFLELQLLNEHLLGHLEQAKSLPPGSQSEAAAPEKEQDTPRGEPPAVPESENVATKEQKKKPRRGRKPKVSKAEQPLVIVEDKEPTEQVAEIITEVPPDEPVSATPDERIMELVLGKLATTTTDTSSVPKFTHHQNNTITLKRSLILSSRHGIRRKLIKQLGEHKRVYQCNICSKIFQNSSNLSRHVRSHGDKLFKCEECAKLFSRKESLKQHVSYKHSRNEVDGEYRYRCGTCEKTFRIESALEFHNCRTDDKTFQCEMCFRFFSTNSNLSKHKKKHGDKKFACEVCSKMFYRKDVMLDHQRRHLEGVRRVKREDLEAGGENLVRYKKEPSGCPVCGKVFSCRSNMNKHLLTHGDKKYTCEICGRKFFRVDVLRDHIHVH

SEQ ID NO: 15 - DNA, 4376 bp, Homo sapiens
  CDS (64)...(3837)
gaagatagtgtgtggctgcttctggactcaaggaggaggagagagattccgcgagccgacaccatgcgatccaaggcgagggcgaggaagctagccaaaagtgacggtgacgttgtaaataatatgtatgagcccaaccgggacctgctggccagccacagcgcggaggacgaggccgaggacagtgccatgtcgcccatccccgtggggtcaccgccccccttccccaccagcgaggacttcacccccaaggagggctcgccgtacgaggcccctgtctacattcctgaagacattccgatcccagcagacttcgagctccgagagtcctccatcccaggggctggcctgggggtctgggccaagaggaagatggaagccggggagaggctgggcccctgcgtggtggtgccccgggcggcggcaaaggagacagacttcggatgggagcaaatactgacggacgtggaagtgtcgccccaggaaggctgcatcacaaagatctccgaagacctgggcagtgagaagttctgcgtggatgcaaatcaggcgggggctggcagctggctcaagtacatccgtgtggcgtgctcctgcgatgaccagaacctcaccatgtgtcagatcagtgagcaggtaatttactataaagtcattaaggacattgagccaggtgaggagctgctggtgcacgtgaaggaaggcgtctaccccctgggcacagtgccgcccggcctggacgaggagcccacgttccgctgtgacgagtgtgacgaactcttccagtccaagctggacctgcggcgccataagaagtacacgtgtggctcagtgggggctgcgctctacgagggcctggctgaggagctcaagcccgagggccttggcggtggcagcggccaagcccacgagtgcaaggactgcgagcggatgttccccaacaagtacagcctggagcagcacatggtcatccacacggaggagcgcgagtacaaatgcgaccagtgtcccaaggccttcaactggaagtccaacttcatccgccaccagatgtcccacgacagcggcaaacgcttcgaatgtgaaaactgcgtgaaggtgttcacggaccccagcaaccttcagcggcacatccgctcgcagcacgtgggcgctcgggcccacgcctgccccgactgcgggaagaccttcgccacgtcctccggcctcaagcagcacaagcatatccacagcacggtgaagcctttcatatgtgaggtctgccacaagtcctacacgcagttctccaacctgtgccggcacaagcggatgcacgccgactgccgcacgcagatcaagtgcaaggactgtggccagatgttcagcactacctcctccctcaacaagcaccggcgcttctgcgagggcaagaaccattacacgccgggcggcatctttgccccgggcctgcccttgacccccagccccatgatggacaaggcaaaaccctcccccagcctcaatcacgccagcctgggcttcaacgagtactttccctacaggccgcacccggggagcctgcccttctccacggcgcctcccacgttccccgcactcacccccggcttcccgggcatcttccctccatccttgtacccccggccgcctctgctacctcccacatcgctgctcaagagccccctgaaccacacccaggacgccaagctccccagtcccctggggaacccagccctgcccctggtctccgccgtcagcaacagcagccagggcacgacggcagctgcggggcccgaggagaagttcgagagccgcctggaggactcctgtgtggagaagctgaagaccaggagcagcgacatgtcggacggcagtgactttgaggacgtcaacaccaccacggggaccgacctggacacgaccacggggacgggctcggacctggacagcgacgtggacagcgaccctgacaaggacaagggcaagggcaagtccgccgagggccagcccaagtttgggggcggcttggcgcccccgggggccccgaacagcgtggccgaggtgcctgtcttctattcccagcactcattcttcccgccacccgacgagcagctgctgactgcaacgggcgccgccggggactccatcaaggccatcgcatccattgccgagaagtactttggccccggcttcatggggatgcaggagaagaagctgggctcgctcccctaccactcggcgttccccttccagttcctgcccaacttcccccactccctttaccccttcacggaccgagccctcgcccacaacttgctggtcaaggccgagccaaagtcaccccgggacgccctcaaggtgggcggccccagtgccgagtgcccctttgatctcaccaccaagcccaaagacgtgaagcccatcctgcccatgcccaagggcccctcggcccccgcatccggcgaggagcagccgctggacctgagcatcggcagccgggcccgtgccagccaaaacggcggcgggcgggagccccgcaagaaccacgtctatggggaacgcaagctgggcgccggcgaggggctgccccaggtgtgcccggcgcggatgccccagcagcccccgctccactacgccaagccctcgcccttcttcatggaccccatctacagggtagaaaagcggaaggtcacagaccccgtgggagccctgaaggagaagtacctgcggccgtccccgctgctcttccacccccagatgtcagccatagagaccatgacagagaagctggagagctttgcagccatgaaggcggactcgggcagctccctgcagcccctcccccaccaccccttcaacttccggtccccacccccaacgctctccgaccccatcctcaggaagggcaaggagcgatacacgtgcaggtactgtgggaagatcttccccagatcagccaatctcaccagacacctgaggacgcacactggggagcagccgtacaggtgtaagtactgcgaccgctccttcagcatctcttcgaacctccagcggcacgtccggaacatccacaacaaggagaagcctttcaagtgccacctgtgcaaccgctgcttcgggcagcagaccaacctggaccggcacctcaagaagcacgagcacgagaacgcaccagtgagccagcaccccggggtcctcacgaaccacctggggaccagcgcgtcctctcccacctcagagtcggacaaccacgcacttttagacgagaaagaagactcttatttctcggaaatcagaaactttattgccaatagtgagatgaaccaagcatcaacgcgaacagagaaacgggcggacatgcagatcgtggacggcagtgcccagtgtccaggcctagccagtgagaagcaggaggacgtggaggaggaggacgacgatgacctggaggaggacgatgaggacagcctggccgggaagtcgcaggatgacaccgtgtcccccgcacccgagccccaggccgcctacgaggatgaggaggatgaggagccagccgcctccctggccgtgggctttgaccacacccgaaggtgtgctgaggaccacgaaggcggtctgttagctttggagccgatgccgacttttgggaaggggctggacctccgcagagcagctgaggaagcatttgaagttaaagatgtgcttaattccaccttagattctgaggctttaaaacatacactgtgcaggcaggctaagaaccaggcatatgcaatgatgctgtccctttccgaagacactcctctccacaccccctcccagggttctctggacgcttggttgaaggtcactggagccacgtcggagtctggagcatttcaccccatcaaccacctctgacgggctgggcagccgggggccggtggccagagcgagggcaccagccacgaaggacggaggcgggcggggccccggagaaccctgtccctgcgtgtggccactcctcagcatcctccccacccaccatggttcattccgacttttccaatggaaactcagatcccaaaagtccctaaagcagtcgtagagtctcaccatctccaaggattggtcttgagaacactgttcagtgacggccatgcaggtggccgtccaaagacagccaacggagctgcctcgcagaatcagccagtgggcaggtggacgctctgctgagacagaagctggtggccactgccgggtgcccgcgtggggtcgcggaagggaatggatagactggtgtgctcaaaagagagagatcactcaaatgatttttataatgaaatgacaagaataacccttttggtaaccgtattgactgcagagtctatttaagcatgtgg

SEQ ID NO: 16 - protein, 1258 aa, Homo sapiens
MRSKARARKLAKSDGDVVNNMYEPNRDLLASHSAEDEAEDSAMSPIPVGSPPPFPTSEDFTPKEGSPYEAPVYIPEDIPIPADFELRESSIPGAGLGVWAKRKMEAGERLGPCVVVPRAAAKETDFGWEQILTDVEVSPQEGCITKISEDLGSEKFCVDANQAGAGSWLKYIRVACSCDDQNLTMCQISEQVIYYKVIKDIEPGEELLVHVKEGVYPLGTVPPGLDEEPTFRCDECDELFQSKLDLRRHKKYTCGSVGAALYEGLAEELKPEGLGGGSGQAHECKDCERMFPNKYSLEQHMVIHTEEREYKCDQCPKAFNWKSNFIRHQMSHDSGKRFECENCVKVFTDPSNLQRHIRSQHVGARAHACPDCGKTFATSSGLKQHKHIHSTVKPFICEVCHKSYTQFSNLCRHKRMHADCRTQIKCKDCGQMFSTTSSLNKHRRFCEGKNHYTPGGIFAPGLPLTPSPMMDKAKPSPSLNHASLGFNEYFPYRPHPGSLPFSTAPPTFPALTPGFPGIFPPSLYPRPPLLPPTSLLKSPLNHTQDAKLPSPLGNPALPLVSAVSNSSQGTTAAAGPEEKFESRLEDSCVEKLKTRSSDMSDGSDFEDVNTTTGTDLDTTTGTGSDLDSDVDSDPDKDKGKGKSAEGQPKFGGGLAPPGAPNSVAEVPVFYSQHSFFPPPDEQLLTATGAAGDSIKAIASIAEKYFGPGFMGMQEKKLGSLPYHSAFPFQFLPNFPHSLYPFTDRALAHNLLVKAEPKSPRDALKVGGPSAECPFDLTTKPKDVKPILPMPKGPSAPASGEEQPLDLSIGSRARASQNGGGREPRKNHVYGERKLGAGEGLPQVCPARMPQQPPLHYAKPSPFFMDPIYRVEKRKVTDPVGALKEKYLRPSPLLFHPQMSAIETMTEKLESFAAMKADSGSSLQPLPHHPFNFRSPPPTLSDPILRKGKERYTCRYCGKIFPRSANLTRHLRTHTGEQPYRCKYCDRSFSISSNLQRHVRNIHNKEKPFKCHLCNRCFGQQTNLDRHLKKHEHENAPVSQHPGVLTNHLGTSASSPTSESDNHALLDEKEDSYFSEIRNFIANSEMNQASTRTEKRADMQIVDGSAQCPGLASEKQEDVEEEDDDDLEEDDEDSLAGKSQDDTVSPAPEPQAAYEDEEDEEPAASLAVGFDHTRRCAEDHEGGLLALEPMPTFGKGLDLRRAAEEAFEVKDVLNSTLDSEALKHTLCRQAKNQAYAMMLSLSEDTPLHTPSQGSLDAWL

SEQ ID NO: 17 - DNA, 2442 bp, Homo sapiens
  CDS (232)...(2391)
agctttcccagctagtcgaatcactggtgcacctccggaatccgaggttcgcattgctctcggacagagttctgcctccccagtactcccaaactcccagtcctgtgcaccaatgaggtccagctctgaggcttctttcctgagaaaaaaatttggaagtccgtgactgtttcctggaggagctgagaagaggaagctcacttccggcgtagggaggctttctgacccggaatggaggaggcggaggagctgctcttggaggggaagaaggcgctgcaactcgcccgcgagccgcgcctgggcctggacttaggatggaacccttccggagaaggctgtacgcagggcctcaaagacgtcccacccgagccgacccgagacatcctcgctttaaagagccttccccggggcttggcccttggcccctcactcgccaaggaacagcgcttgggggtctggtgtgtcggggaccccctgcagcccggcctgctgtgggggccgctggaagaggagtctgcctccaaggagaagggcgagggagtaaagccacggcaggaggagaacctgtcattaggcccatggggagacgtgtgtgcctgtgagcagagttctggctggactagcttggtacaacggggcaggctggagagtgagggaaatgtggccccagtgcggatcagcgagaggcttcatctgcaagtgtaccagctggtgctgccaggctctgaactgctgctgtggccccagccttcctctgagggcccaagtctcacccagcctgggctggacaaagaggcagctgtagcagtggtgacagaagtggagtctgctgtacagcaggaagtggcctcccctggggaggatgcagcagaaccttgcatagatcctggttcccagtcaccctctggcatccaggcagagaatatggtgagccctggacttaagttcccaacccaggaccgaatttccaaggatagccagccacttggcccattgcttcaggatggcgacgtggatgaggaatgcccggcccaggcacagatgccacctgaacttcagagcaattcggctacccagcaggacccagatggcagtggagccagtttctcatcttctgccaggggcacccagccgcatggctacctggccaagaagttacacagccccagtgatcagtgcccacccagagcaaagaccccagagcctggagcccagcagtctggcttccctacactctcgcggagccctcctggcccagcaggaagctccccaaagcaggggcgacggtaccggtgtggagagtgtggcaaggcattcctacagctgtgccacctaaagaagcacgcatttgtgcacacgggccacaagccctttctttgcactgagtgtggcaagagctatagctcagaggagagcttcaaagcccatatgctgggccaccgtggggtgcggcccttcccctgtccacaatgcgacaaggcctatggcacccagcgagacctcaaagagcaccaggtggtacattcaggtgcccggccctttgcttgtgaccagtgtggcaaggcctttgcccgccggccctccctgcggctgcatcgcaagacccaccaggtgccagctgcccctgccccttgcccatgccctgtgtgtgggcggcccctggccaaccagggctccctgcggaaccatatgaggctccatacaggagaaaagcctttcctgtgcccgcactgtggccgggcgtttcgtcagcggggcaacctgcgtgggcatttgcggctccacaccggggagcgtccttaccgctgcccacactgtgccgatgccttcccccagctgcctgaactgcggcgccatctcatctcacacaccggggaggcccacttgtgcccggtgtgtggcaaggccctccgagacccacacacgctccgagctcacgagcgtctgcactccggagagaggccctttccctgtccccagtgtggccgtgcttacacgctggccaccaagctgcggcgccacctcaaatctcacttggaggacaagccctaccgctgccccacctgtggcatgggctacaccctcccgcagagcctcaggcggcatcagctcagtcaccggcctgaggcaccctgcagcccaccctctgtgccttctgctgcttctgagcccactgtggtgctcctgcaggctgagccacaactgctggacacacacagagaggaggaagtctcccccgccagggatgttgttgaggtcaccatttcagaaagccaggagaagtgctttgtggtgccagaggagccagatgccgcccccagcctggtgctaatccataaggacatgggcctcggcgcctgggcagaggtggtggaggtggagatgggcacctgacagctttgccttttgctgacacagctccataaagactcgtgctttctc

SEQ ID NO: 18 - protein, 720 aa, Homo sapiens
MEEAEELLLEGKKALQLAREPRLGLDLGWNPSGEGCTQGLKDVPPEPTRDILALKSLPRGLALGPSLAKEQRLGVWCVGDPLQPGLLWGPLEEESASKEKGEGVKPRQEENLSLGPWGDVCACEQSSGWTSLVQRGRLESEGNVAPVRISERLHLQVYQLVLPGSELLLWPQPSSEGPSLTQPGLDKEAAVAVVTEVESAVQQEVASPGEDAAEPCIDPGSQSPSGIQAENMVSPGLKFPTQDRISKDSQPLGPLLQDGDVDEECPAQAQMPPELQSNSATQQDPDGSGASFSSSARGTQPHGYLAKKLHSPSDQCPPRAKTPEPGAQQSGFPTLSRSPPGPAGSSPKQGRRYRCGECGKAFLQLCHLKKHAFVHTGHKPFLCTECGKSYSSEESFKAHMLGHRGVRPFPCPQCDKAYGTQRDLKEHQVVHSGARPFACDQCGKAFARRPSLRLHRKTHQVPAAPAPCPCPVCGRPLANQGSLRNHMRLHTGEKPFLCPHCGRAFRQRGNLRGHLRLHTGERPYRCPHCADAFPQLPELRRHLISHTGEAHLCPVCGKALRDPHTLRAHERLHSGERPFPCPQCGRAYTLATKLRRHLKSHLEDKPYRCPTCGMGYTLPQSLRRHQLSHRPEAPCSPPSVPSAASEPTVVLLQAEPQLLDTHREEEVSPARDVVEVTISESQEKCFVVPEEPDAAPSLVLIHKDMGLGAWAEVVEVEMGT

SEQ ID NO: 19 - DNA, 1431 bp, Homo sapiens
  CDS (151)...(1185)
ctaagaaacaaaagaatttcaagataattaggtggagcgggcgggctggctgctgaggacgcgccgcctgcgccttcctccctgcgtgcctcgccccgggcggcccggggctgccgcggtgcgcgggtgccgggccctgccttgccggccatgggggaagggggcgccgcggcggcgctggtggcggcggcagcagcagcagcagcggcagcggcagcggtggtggccgggcagcggcggcggcggctagggcgcagggcgcgctgccatgggcctggccgggctgcaggcgggaagatgtccaagccctgcgcggtggaggcggcggcggcggcggtggcagcgacggccccgggcccggagatggtggagcggaggggcccggggaggccccgcaccgacggggagaacgtatttaccgggcagtcaaagatctattcctacatgagcccgaacaaatgctctggaatgcgtttcccccttcaggaagagaactcagttacacatcacgaagtcaaatgccaggggaaaccattagctggaatctacaggaaacgagaagagaaaagaaatgctgggaacgcagtacggagcgccatgaagtccgaggaacagaagatcaaagacgccaggagacgtcccctgaaggaaaaacgcaacagaatcgcaaacacggatttctaccctgtccgaaggagctccaggaagagcaaagccgagctgcagtctgaagaaaggaaaagaatagatgaattgattgaaagtgggaaggaagaaggaatgaagatcgacctcatcgatggcaaaggcaggggtgtgattgccaccaagcagttctcccggggtgcctttgtggtggaataccacggggacctcatcgagatcaccgacgccaagaaacgggaggctctgtacgcacaggacccttccacgggctgctacatgtactattttcagtatctgagcaaaacctactgcgtggatgcaactagagagacaaatcgcctaggaagactgatcaatcacagcaaacgtgggaactgccaaaccaaactgcacgacatcgacggcgtacctcacctcatcctcatcgcctcccgagacatcgcggctggggaggagcccctgtatgactatggggaccgcagcaaggcttccattgaagcccacccatggctgaagcattaaccggtgggccccgcgccctccccgccccactttcccttcttcaaaggacaaagtgccctcaaagggaattgaatttttttttacacacttaatcttagcggattacttcagatgtttttaaaaagtatattaagatgccttttcactgtagtatttaaatatctgttacaggtttccaaggtggacttgaacagatggccttatattaccaaaacttttatattctagttgtttttgtacttt

SEQ ID NO: 20 - protein, 345 aa, Homo sapiens
MGEGGAAAALVAAAAAAAAAAAAVVAGQRRRRLGRRARCHGPGRAAGGKMSKPCAVEAAAAAVAATAPGPEMVERRGPGRPRTDGENVFTGQSKIYSYMSPNKCSGMRFPLQEENSVTHHEVKCQGKPLAGIYRKREEKRNAGNAVRSAMKSEEQKIKDARRRPLKEKRNRIANTDFYPVRRSSRKSKAELQSEERKRIDELIESGKEEGMKIDLIDGKGRGVIATKQFSRGAFVVEYHGDLIEITDAKKREALYAQDPSTGCYMYYFQYLSKTYCVDATRETNRLGRLINHSKRGNCQTKLHDIDGVPHLILIASRDIAAGEEPLYDYGDRSKASIEAHPWLKH

SEQ ID NO: 21 - DNA, 26 bp, Artificial Sequence
  Primer
tgtccctgcacgcccggaagtagatg

SEQ ID NO: 22 - DNA, 22 bp, Artificial Sequence
  Primer
tgtgctggaacgccagcaggtt

SEQ ID NO: 23 - DNA, 23 bp, Artificial Sequence
  Primer
gggggtagacgccttggttcacg

SEQ ID NO: 24 - DNA, 21 bp, Artificial Sequence
  Primer
catcgcaggagcacgccacac

SEQ ID NO: 25 - DNA, 967 bp, Homo sapiens
gtcttgaggaccatctctcccggcagcataccgtgtggcttcacactgctctgcctctctgaacctcggtttcttcatctataaaatgggaataagagtaagccacctcaatggactgtgggaggcttaagtaaattgaagtgccatgcaagtagctagcatgcagttgcagctcaatgaatattatgatggccgcagatacgatggctacagctggggcacccatttcgggtcacaaggtagggttcaatgttgaagatggcagagccaattgcatccctgatgatcgtggagtgccgggcctgcctgagatgctcacctctcttcctttaccagagagagaaagacagaatgaccgagaacatgaaggagtgcttggcccagaccaatgcagccgtgggggatatggtgacggtggtgaatccgagccaggagtatggccagccctgctctaggagaccggactcctcggccatggaagttgagcccaagaaactgaaagggaagcgcgacctcatcgtgcccaaaagcttccagcaagtggacttctggttttgtgagtcctgccaggagtacttcgtggatgaatgcccaaaccatggccccccggtgtttgtgtctgacacaccggtgcccgtgggcatcccagaccgggcggcgctcaccatcccacagggcatggaggtggtcaaggacactagtggagagagtgacgtgcgatgtgtaaacgaggtcatccccaagggccacatcttcggcccctatgaggggcagatctccacccaggacaaatcagctggcttcttctcctggctgattgtggacaagaacaaccgctataagtccatagatggctcagacgagaccgaagccaactggatgaggtacgtggtcatctcccgggaggagagggagcagaacctgctggcgttccagcacagtgagcgcatctacttccgggcgtgca

SEQ ID NO: 26 - DNA, 622 bp, Homo sapiens
gaagatagtgtgtggctgcttctggactcaaggaggaggagagagattccgcgagccgacaccatgcgatccaaggcgagggcgaggaagctagccaaaagtgacggtgacgttgtaaataatatgtatgagcccaaccgggacctgctggccagccacagcgcggaggacgaggccgaggacagtgccatgtcgcccatccccgtggggtcaccgccccccttccccaccagcgaggacttcacccccaaggagggctcgccgtacgaggcccctgtctacattcctgaagacattccgatcccagcagacttcgagctccgagagtcctccatcccaggggctggcctgggggtctgggccaagaggaagatggaagccggggagaggctgggcccctgcgtggtggtgccccgggcggcggcaaaggagacagacttcggatgggagcaaatactgacggacgtggaagtgtcgccccaggaaggctgcatcacaaagatctccgaagacctgggcagtgagaagttctgcgtggatgcaaatcaggcgggggctggcagctggctcaagtacatccgtgtggcgtgctcctgcgatgaccagaacctcaccatgtgtc

SEQ ID NO: 27 - protein, 114 aa, Homo sapiens
KTRIFVWATKPILKGKKFGPEVGDKKKRSQVKNNVYMWEVYYPNLGWMCIDATDPEKGNWLRYVNWACSGEEQNLFPLEINRAIYYKTLKPIAPGEELLVWYNGEDNPEIAAAI

SEQ ID NO: 28 - protein, 117 aa, Homo sapiens
EEVIGVMSKEYIPKGTRFGPLIGEIYTNDTVPKNANRKYFWRIYSRGELHHFIDGFNEEKSNWMRYVNPAHSPREQNLAACQNGMNIYFYTIKPIPANQELLVWYCRDFAERLHYPY

SEQ ID NO: 29 - protein, 111 aa, Homo sapiens
GAGLGIWTKRKIEVGEKFGPYVGEQRSNLKDPSYGWEILDEFYNVKFCIDASQPDVGSWLKYIRFAGCYDQHNLVACQINDQIFYRVVADIAPGEELLLFMKSEDYPHETM

SEQ ID NO: 30 - protein, 117 aa, Homo sapiens
IHGRGLFCKRNIDAGEMVIEYAGNVIRSIQTDKREKYYDSKGIGCYMFRIDDSEVVDATMHGNRARFINHSCEPNCYSRVINIDGQKHIVIFAMRKIYRGEELTYDYKFPIEDASNK

SEQ ID NO: 31 - protein, 119 aa, Homo sapiens
IHNWGLYALDSIAAKEMIIEYVGERIRQPVAEMREKRYLKNGIGSSYLFRVDENTVIDATKKGGIARFINHCCDPNCTAKIIKVGGRRRIVIYALRDIAASEELTYDYKFEREKDDEER

SEQ ID NO: 32 - protein, 89 aa, Homo sapiens
GSGWGVRAATALRKGEFVCEYIEEIITSDEANERGKAYDDNGRTYLFDLDYNTAQDSEYTIDAANYGNISHFINHFDYIRADNEDVPYE

SEQ ID NO: 33 - protein, 118 aa, Homo sapiens
DKGWGVRTKLPIAKGTYILEYVGEVVTEKEFKQRMASIYLNDTHHYCLHLDGGLVIDGQRMGSDCRFVNHSCEPNCEMQKWSVNGLSRMVLFAKRAIEEGEELTYDYNFSLFNPSEGQ

(19) United States
(12) Patent Application Publication
(10) Pub. No.: US 2003/0049623 A1
(43) Pub. Date: Mar. 13, 2003

(54) PR/SET-domain containing nucleic acids, polypeptides, antibodies and methods of use

(76) Inventor: Shi Huang, San Diego, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/910,478

(22) Filed: Jul. 18, 2001

(51) Int. Cl. C12Q 1/68; C07H 21/04; C12P 21/02; C12N 5/06

(52) U.S. Cl. 435/6; 435/455; 435/325; 435/320.1; 435/69.1; 530/350; 536/23.2

(57) ABSTRACT

The present invention provides an isolated nucleic acid molecule encoding a PFM/SET polypeptide. Also provided is an isolated nucleic acid molecule encoding a functional fragment of a PFM/SET polypeptide that contains a PR, SET, PRAZ or PKZL domain of a PFM/SET polypeptide of the invention. Further provided by the invention are PFM/SET polypeptides, and functional fragments thereof that contain a PR, SET, PRAZ or PKZL domain of a PFM/SET polypeptide. The invention also provides PFM/SET antibodies, PFM/SET modulatory compounds, and related methods. The molecules and methods of the invention can be used to modulate cell proliferation to prevent or treat proliferative disorders, including cancer. Additionally, the molecules and methods of the invention can be used to diagnose and prognose proliferative disorders.

This invention was made in part with government support under grant number CA67146, awarded by the National Institutes of Health. Accordingly, the United States government has certain rights in this invention.

### BACKGROUND OF THE INVENTION

1Field of the Invention

This invention relates generally to proliferative disorders such as cancer and, more specifically, to PR/SET-domain containing genes and gene products that can be used to diagnose and treat proliferative disorders.

2Background Information

Cancer is one of the leading causes of death in industrialized nations. Cancerous tumors result when a cell escapes from its normal growth regulatory mechanisms and proliferates in an uncontrolled fashion. Cells from the primary tumor generally metastasize to vital organs if treatment of the primary tumor is either not complete or not initiated early enough. Thus, early diagnosis and effective treatment of tumors is essential for survival.

Cancer involves the clonal replication of populations of cells that have gained competitive advantage over normal cells through the alteration of regulatory genes. Regulatory genes can be broadly classified into “oncogenes” which, when activated or overexpressed promote unregulated cell proliferation, and “tumor suppressor genes” which, when inactivated or underexpressed fail to prevent abnormal cell proliferation. Loss of function or inactivation of tumor suppressor genes is thought to play a central role in the initiation and progression of a significant number of human cancers.

A number of tumor suppressor genes have been identified that, when inactivated, are involved in the initiation or progression of human cancers. Known tumor suppressor genes include RB, p53, DCC, APC/MCC, RIZ, NF1, NF2, WT1, VHL, BRCA1, MST1 and WAF1/CIP1. Approaches for treating cancer by modulating the function of several of these tumor suppressor genes, either with pharmaceutical compounds that target their encoded proteins, or by gene therapy methods, have yielded promising results in animal models and in human clinical trials.

Approaches for diagnosing and prognosing cancer by identifying mutations in tumor suppressor genes have also been developed. For example, identifying individuals containing germline mutations in known tumor suppressor genes has permitted the identification of individuals at increased risk of developing cancer. Such individuals are then closely monitored or treated prophylactically to improve their chance of survival. Identifying the pattern of alterations of known tumor suppressor genes in biopsy samples is also being used to determine the presence or stage of a tumor. Being able to determine whether a cancer is benign or malignant, or at an early or late stage of progression, provides the patient and clinician with a more accurate prognosis and can be used to determine and monitor the course of treatment.

One important family of tumor suppressor genes that has recently been identified are PR/SET-domain containing genes. PR and SET domains are structurally related motifs present in proteins that function in modulating gene activities from yeast to mammals. A PR domain is a motif first identified as a region of homology between the Rb-binding zinc finger protein RIZ, and the transcriptional repressor protein PRDI-BFI/Blimpl, which promotes B-cell differentiation (Buyse et al., Proc. Natl. Acad. Sci. USA 92:4467-4471 (1995); Huang, Cell 78:9 (1994)). A PR domain motif is also found in the MDS1-EVI1 myeloid leukemia gene (Fears et al., Proc. Natl. Acad. Sci. USA 93:1642-1647 (1996)). A SET domain is a motif first identified as a region of homology between the Drosophila melanogaster genes Su(var)3-9, Enhancer-of-zeste and Trithorax. PR and SET domain-encoding genes have also been identified in other mammals and in lower organisms, including C. elegans, suggesting an evolutionarily conserved function for these domains.

In view of the importance of tumor suppressor genes and related molecules in the detection and treatment of cancer, there exists a need to identify additional tumor suppressor genes. In particular, in view of the established role of PR/SET-domain containing genes as tumor suppressor genes, there exists a need to identify and characterize additional PR/SET-domain family members. The present invention satisfies this need and provides related advantages as well.

### SUMMARY OF THE INVENTION

The invention provides an isolated nucleic acid molecule encoding a PFM/SET polypeptide that has at least about 80% identity with an amino acid sequence of an invention PFM/SET polypeptide.

The invention also provides an isolated nucleic acid molecule having a nucleotide sequence that encodes a functional fragment of a PFM/SET polypeptide, the functional fragment containing a PR, SET, PRAZ, or PKZL domain of a PFM/SET amino acid sequence of the invention.

Further provided is an isolated oligonucleotide containing at least 17 contiguous nucleotides of an invention PFM/SET nucleotide sequence, or the complement thereof.

Also provided are methods for detecting a PFM/SET nucleic acid molecule in a sample. In one embodiment, the method involves contacting a sample with the PFM/SET nucleic acid molecule under conditions that allow specific hybridization to PFM/SET nucleic acid, and detecting said specific hybridization. In another embodiment, the method is practiced by contacting a sample with a PFM/SET primer pair under conditions that allow amplification of PFM/SET nucleic acid, and detecting amplified PFM/SET nucleic acid.

Further provided is a method for modulating cell growth. The method involves introducing a vector containing an invention PFM/SET nucleic acid molecule into a host cell, and expressing encoded PFM/SET polypeptide in an amount effective to modulate growth of said cell.

The invention also provides an isolated PFM/SET polypeptide, containing a PFM/SET amino acid sequence having at least about 80% amino acid identity with an invention PFM/SET amino acid sequence.

Further provided by the invention is a functional fragment of a PFM/SET polypeptide, the functional fragment containing a PR, SET, PRAZ, or PKZL domain of a PFM/SET amino acid sequence of the invention

Also provided is an isolated immunogenic PFM/SET peptide containing at least 8 contiguous amino acids of an invention PFM/SET amino acid sequence.

The invention also provides an antibody, or antigen binding fragment thereof, which specifically binds to a PFM/SET polypeptide of the invention.

Further provided is a method for detecting PFM/SET polypeptide in a sample. The method is practiced by contacting a sample with the antibody under conditions that allow specific binding of said antibody to PFM/SET polypeptide, and detecting specifically bound antibody.

### BRIEF DESCRIPTION OF THE DRAWINGS

FIG. 1 shows a comparison of the amino acid sequences of the PR domains of RIZ1 (SEQ ID NO:27), BLIMP1 (SEQ ID NO:28), MDS1-EVI1 (SEQ ID NO:29), HRX (ALL-1)(SEQ ID NO:30), and the SET domains of SET1 (SEQ ID NO:31), Su(var)3-9 (SEQ ID NO:32), and ASH1 (SEQ ID NO:33).

FIG. 2 shows the amino acid sequence of PFM6 (SEQ ID NO:2). Underlined sequences are the 5′ boundaries of each zinc finger motif.

FIG. 3 shows the amino acid sequence of PFM7 (SEQ ID NO:4) . Underlined sequences are the 5′ boundaries of each zinc finger motif.

FIG. 4 shows the amino acid sequence of PFM9 (SEQ ID NO:8) . Underlined sequences are the 5′ boundaries of each zinc finger motif.

FIG. 5 shows the amino acid sequence of PFM10 (SEQ ID NO:10). Underlined sequences are the 5′ boundaries of each zinc finger motif.

FIG. 6 shows the amino acid sequence of PFM11 (SEQ ID NO:12). Underlined sequences are the 5′ boundaries of each zinc finger motif.

FIG. 7 shows the amino acid sequence of PFM12 (SEQ ID NO:14). Underlined sequences are the 5′ boundaries of each zinc finger motif.

FIG. 8 shows the amino acid sequence of PFM13 (SEQ ID NO:16). Underlined sequences are the 5′ boundaries of each zinc finger motif.

FIG. 9 shows the amino acid sequence of PFM14 (SEQ ID NO:18). Underlined sequences are the 5′ boundaries of each zinc finger motif.

### DETAILED DESCRIPTION OF THE INVENTION

The present invention provides “PFM/SET” nucleic acid molecules, polypeptides, antibodies, modulatory compounds, and related methods. “PFM/SET” nucleic acid molecules and polypeptides contain one or more “PR” or “SET” domains, which are structurally and functionally related nucleotide and amino acid motifs. The PFM and SET nucleic acids and polypeptides of the invention are important regulators of cell proliferation. Therefore, the molecules and methods of the invention can be used to modulate cell proliferation so as to prevent or treat proliferative disorders, including cancer. Additionally, the molecules and methods of the invention can be used to diagnose and prognose proliferative disorders.

PFM/SET nucleic acid molecules and polypeptides are generally characterized by encoding or containing a PR domain or SET domain. Many PFM/SET nucleic acid molecules and polypeptides also encode or contain a zinc finger motif, and can contain other motifs. Exemplary PFM, or “PR family member” nucleic acid molecules include RIZ, MDS1-EVI1 and PRDI-BF1 (BLIMP1). Exemplary SET nucleic acid molecules include HRX/ALL1/MLL and MMSET.

PR and SET domains have been demonstrated to be protein binding motifs involved in the regulation of gene expression (Huang et al., J. Biol. Chem. 273:15933-15940 (1998); Soderholm et al., Leukemia 11:352-358 (1997)). In particular, the domains are considered to function in the assembly of chromatin-based multiprotein complexes involved in either euchromatin-mediated gene activation, or heterochromatin-mediated gene silencing. Lack or inactivation of the PR or SET domain can thus specifically inactivate the chromatin-associated functions of a PFM/SET polypeptide, without affecting other activities such as DNA binding and chromatin-independent transcriptional activation or repression. PFM genes encoding PR lacking isoforms of PFM polypeptides have been characterized. Differential chromatin regulation by the PR+ and PR− forms of a PFM gene may underlie the opposite roles of these products in tumorigenesis.

The PR domain is about 100 to 125 amino acids in length, and contains three highly conserved sequences, designated the A, B and C boxes, each of which consists of about 6 to 12 amino acids. Each of boxes A, B and C is encoded by a separate exon. Alternative exon usage of the A, B and C boxes, and transcription from internal promoters, can result in PFM transcripts and polypeptides containing all, some or none of the A, B or C boxes.

Several observations indicate that PR-domain containing gene products are negative regulators of cell growth and tumorigenesis, whereas the PR-deficient products of these genes are involved in growth promotion and oncogenesis. For example, the PR region of MDS1-EVI1 is often disrupted by leukemia-associated chromosomal insertions and translocations. These disruptions result in loss of the PR-containing MDS1-EVI1 product and selective retention of the PR-deficient EVI1 product. In contrast, the PR- EVI1 product is overexpressed in some tumor cells, and acts as an oncogene (Morishita et al., Cell 54:831-840 (1988); Morishita et al., Proc. Natl. Acad. Sci. USA 89:3937-3941 (1992))

Similarly, the RIZ gene produces two products, a PR-containing protein, RIZ1, and a PR-deficient protein, RIZ2, which is generated from an internal promoter. RIZ1 is commonly absent or underexpressed in a number of human neoplasias, including breast cancer, neuroblastoma and lung cancer. In these cases, the PR-deficient product, RIZ2, is expressed at normal levels (He et al., Cancer Res. 58:4238-4244 (1998)). These results suggest that the PR-containing RIZ1 product is a negative regulator of cell proliferation and tumorigenesis, whereas maintenance of RIZ2 expression may be required for oncogenesis.

As further evidence that RIZ is a tumor suppressor gene, forced expression of the RIZ1 product in breast cancer cells causes cell cycle arrest at the G2/M phase of the cell cycle, and programmed cell death (He et al., Cancer Res. 58:4238-4244 (1998)). Additionally, consistent with a role of RIZ1 in growth suppression, mice in which RIZ1, but not RIZ2, is inactivated, are tumor prone.

In addition, RIZ1 expression is commonly silenced in human cancers, including, for example, breast cancer, liver cancer, colon cancer, neuroblastoma, melanoma, lung cancer and osteosarcoma (He et al., supra (1998) and Jiang et al., Int. J. Cancer 83:541-547 (1999)). RIZ1 gene silencing occurs through methylation of CpG island motifs contained in the promotor region of the RIZ1 gene. RIZ1 deficiency in mice causes formation of B-cell lymphomas and other tumors, as well as accelerated tumor formation in p53 mutant mice. In addition, frequent frame shift mutation of RIZ1 in colorectal tumors with DNA repair defects have been observed (Chadwick et al., Proc. Natl. Acad. Sci. USA 97:2662-2667 (2000), Piao et al., Cancer Res. 60:4701-4704 (2000), and Sakurada et al., Genes, Chromosomes Cancer In press (2000))

Consistent with inactivation of RIZ1 in a broad spectrum of human cancers, recombinant adenovirus-mediated RIZ1 expression can induce G2/M cell-cycle arrest, apoptosis, or both in several tumor cell lines (Chadwick et al., supra, (2000); He et al., supra (1998); Jiang et al., supra, (1999)). RIZ1 can also suppress growth of xenograft colorectal cancers (Jiang and Huang, Histol Histopathol 15:109-117(2000)).

Another PR domain-containing gene, PRDI-BF1/BLIMP1 is also likely to be a tumor suppressor gene. PRDI-BF1/BLIMP1 maps to the 6q21 region commonly deleted in non-Hodgkin's lymphoma (Mock et al., Genomics 37:24-28 (1996)) and is thus a strong candidate tumor suppressor for B-cell non-Hodgkin's lymphoma. Additionally, PRDI-BF1/BLIMP1 is a transcriptional repressor of c-Myc (Lin et al., Science 276:596-598 (1997)), which is an oncogene critically involved in B cell lymphoma.

The PR-domain containing gene PFM1/SC-1 is also likely to be a tumor suppressor gene. PFM1/SC-1 localizes to a tumor suppressor locus and plays a role in promoting cell growth arrest and differentiation induced by serum starvation and nerve growth factor (Chittka and Chao, Proc. Natl. Acad. Sci. USA, 96:10710-10750(1999); Yang and Huang, Genomics 61:319-325 (1999)).

SET nucleic acid molecules and polypeptides are characterized by encoding a “SET domain.” The SET domain is a 150 amino acid motif discovered to be contained in several Drosophila development genes (Jones and Gelbart, Mol. Cell Biol. 13:6357-6366 (1993); Tschiersch et al., EMBO J. 13:3822-3831 (1994)). SET stands for the names of three Drosophila genes which contain the domain, Su(var)3-9, Enhancer-of-zeste and Trithorax. Other exemplary SET nucleic acid molecules include the human trithorax homolog HRX/ALL1/MLL, mutation of which has been observed in human leukemia (Djabali et al., Nature Genetics 2:113-118 (1992; Gu et al., Cell 71:701-708 (1992); Tkachuk et al., Cell 71:691-700 (1992)) and the MMSET gene, mutation of which has been observed in human myeloma (Chesi et al., Blood 92:3025-34 (1998).

Several SET domain containing genes in both Drosophila and mouse function in maintaining gene expression (Yu et al., Proc. Natl. Acad. Sci. USA 95:10632-6 (1998)). In particular, several SET domain containing genes, members of the polycomb and trithorax groups of Drosophila genes, function in sustaining homeobox gene expression by maintaining genes in heterochromatin or euchromatin states (Yu et al., supra, (1998)).

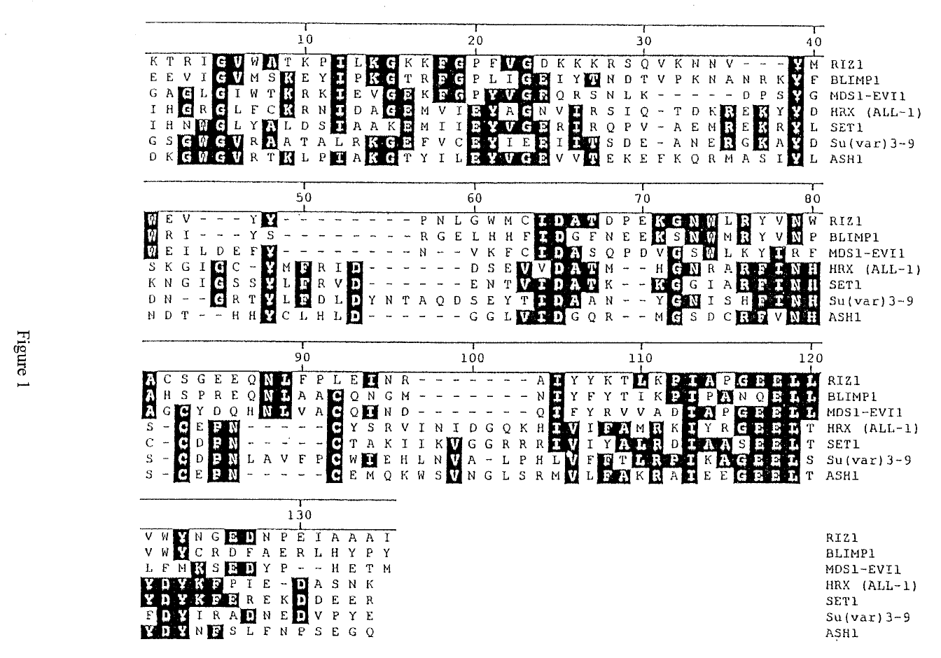

SET and PR domains are structurally and functionally related. These domains share amino acid identity in about 20% of amino acid residues, typically among the most conserved residues in each domain (Huang et al., J. Biol. Chem. 273:15933-15940 (1998)). For example, one of the two conserved leucine residues required for RIZ1 PR protein binding activity is also conserved in the SET domain (Huang et al., supra,(1998)). FIG. 1 shows an amino acid sequence comparison of the PR domains of RIZ1, BLIMP1, MDSa-EVI1, HRX (ALL-1) and the SET domains of SET1, Su(var)3-9, and ASH1. The conserved residues among these domains are highlighted.

Recent data suggest that PR and SET domains are functionally related, both having protein methyltransferase activity that regulates the function of histones and other proteins, particularly during mitosis (Bannister et al., Nature 410:120-124 (2001); Lachner et al., Nature 410:116-120 (2001); Rea et al., Nature 406:593-599 (2000)). PR/SET domain containing genes have also been shown to function in cell memory and in maintaining gene expression patterns in differentiated cells through many cell divisions (Caldas and Aparicio, Cancer & Metastasis Reviews 18:313-29 (1999)). In addition, a recent study in yeast suggests a close relationship between the SET protein SETlp and the checkpoint proteins Mec3p and Rad9p (Corda et al., Nature Genetics 21:204-8 (1999)). The study suggests an important role of the PR/SET domain chromosomal proteins in linking checkpoint to DNA damage in the context of chromatin.

The methyltransferase activity of PR/SET domains contributes to the tumor suppressor function of polypeptides containing this domain. The observation that altered cellular methyltransferase activity is associated with tumorigenesis supports an important role for the methyltransferase activity of PR and SET domains in PFM/SET polypeptide tumor suppressor function. For example, cancer cells commonly lose the enzyme methylthioadenosine phosphorylase (MTAP) (Toohey et al., Biochemical and Biophysical Research Communications, 78:1273-1280 (1977)). An inhibitor of methyltransferases, methylthioadenosine (MTA), commonly accumulates in cancer cells as a result of MTAP deficiency (Nobori, et al. Proc. Natl. Acad. Sci. USA, 93:6203-6208 (1996)). MTA inhibits the aminopropyltransferase enzymes that synthesize polyamines from putrescine and decarboxylated S-adenosylmethionine, and also impairs S-adenosylmethionine dependent trans-methylation reactions.

MTAP is abundant in normal tissues and prevents the inhibition by cleaving MTA to adenine and 5′-methylthioribose L-phosphate, that are recycled to adenine nucleotides and methionine, respectively. MTAP is ˜100 kb apart from the tumor suppressor p16INK4A (CDKN2) on chromosome 9p21, one of the most commonly deleted (homozygous) regions in human cancer. Homozygous deletions at 9p21 commonly involve both genes in many cancers (Toohey et al., supra (1977)). MTAP deletion could facilitate tumor formation and/or progression by causing accumulation of the MTase inhibitor, MTA, which in turn could inactivate RIZ1 and related PFM/SET family of tumor suppressor genes.

In addition to encoding proteins characterized by biological activities consistent with tumor suppressor functions in cells, several PR/SET domain-containing genes are localized to regions of human chromosomes associated with a variety of cancers. Consistent with this observation, genomic sequences corresponding to the isolated PR/SET nucleic acids of the invention have been mapped to chromosomal regions that are altered in human cancers, as summarized in Table 1. The cancers indicated in Table 1 are abbreviated as follows: B-lym—B lymphocytes, Blad—bladder, Co—colon, Eso—esophagus, Fibr(+)—fibrosarcoma, Leu—leukemia, Li—liver, Lu—lung, Ma—mammary, Oral—oral tumor, Ov—ovary, Ov(+)—amplication, Ov(−)—deletion, Pr—prostate, Mel—melanoma, St—stomach, Nc—neurocrest tumor.

TABLE 1

| GENE | CHROMOSOME | CANCER |
|---|---|---|
| PRDM1 | 6q21-q22.1 | B-lym, |
| (PRDIBF1/BLIMP1) | | Mel, St |
| PRDM2 (RIZ1) | 1p36.13-p36.23 | B-lym, Li, |
| | | Ma, Co, |
| | | St, Nc |
| PRDM3 (MDS1-EVI1) | 3q26 | Leu (+/−), |
| | | Ov (+) |
| PRDM4 (PFM1/SC1) | 12q23-24.1 | Pa, Ov, St |
| PRDM5 (PFM2) | 4q25-q26 | Ov, Ma, |
| | | Li, Lu, Co |
| PRDM6 (PFM3) | 5q21-q23 | Co, Lu, Ov |
| | | St, Leu |
| PRDM7 (PFM4) | 16q24 | Ma, Pr, Li |
| PRDM8 (PFM5) | 4q21.1 | Co |
| PRDM9 (PFM6) | 5p14 | Ov (+), |
| | | fibr. (+) |
| PRDM10 (PFM7) | 11q25 | Ma, Co, Ov |
| | | Oral |
| PRDM11 (PFM8) | 11p11.2 | Ma |
| PRDM12 (PFM9) | 9q33-q34.1 | Ov, Blad, |
| | | Eso, lu |
| PRDM13 (PFM10) | 6q16-q21 | B-lym, |
| | | Mel, St |
| PRDM14 (PFM11) | 8p12-21 | Ma, Pr |
| PRDM15 (PFM12) | 21q22.3 | Leu, Blad |
| PRDM16 (PFM13) | 1p36.23-p36.33 | B-lym, Li, |
| | | Ma, Co, |
| | | St, Nc |
| SET07 | 13q11-q13 | Ma |

The PFM/SET nucleic acid molecules and encoded polypeptides of the invention can additionally contain a zinc finger domain, or “ZF domain.” Generally, the ZF domain contains one or several C2H2 or Krüppel-like Zinc finger motifs, which can be represented by the structure: Cys-X2-Cys-X12-His-X3-His. However, a zinc finger motif of a PFM/SET polypeptide can have an amino acid other than Cys or His at one or more of the four conserved positions, and/or alternative spacings between the four conserved positions. The zinc finger motif is found in at least 300 human genes, and is known to specifically bind DNA or RNA sequences (Bellefroid et al., DNA 8:377-387 (1989)).

The PFM/SET nucleic acid molecules of the invention can contain one or more individual zinc finger motifs within a zinc finger domain. For example, PFM6 contains 14 individual zinc finger motifs within the zinc finger domain, while PFM7, PFM13 and PFM14 contain 10 zinc finger motifs, and PFM9, PFM10 and PFM12 contain 3, 4, and 8 zinc finger motifs, respectively.

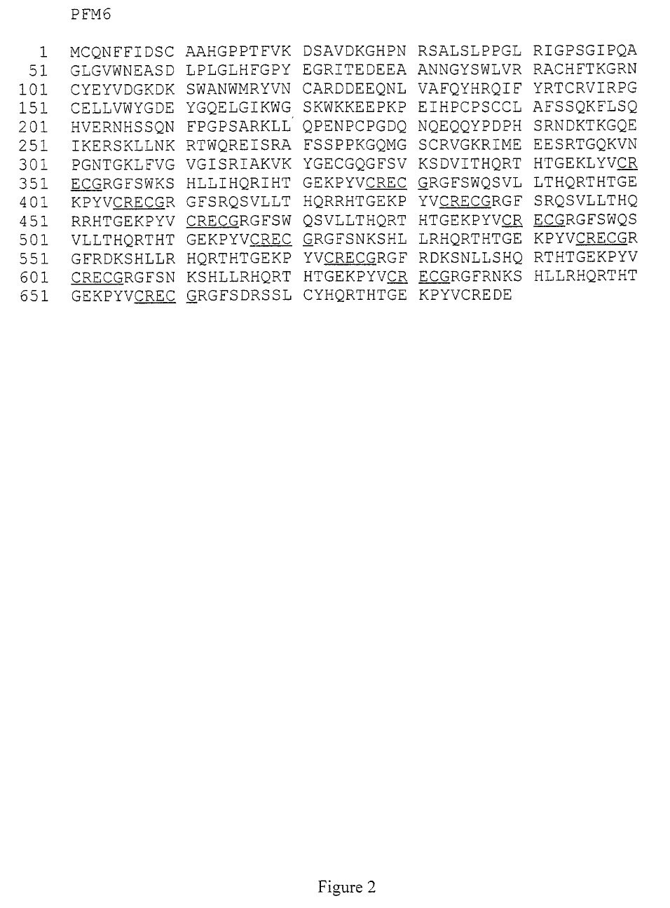

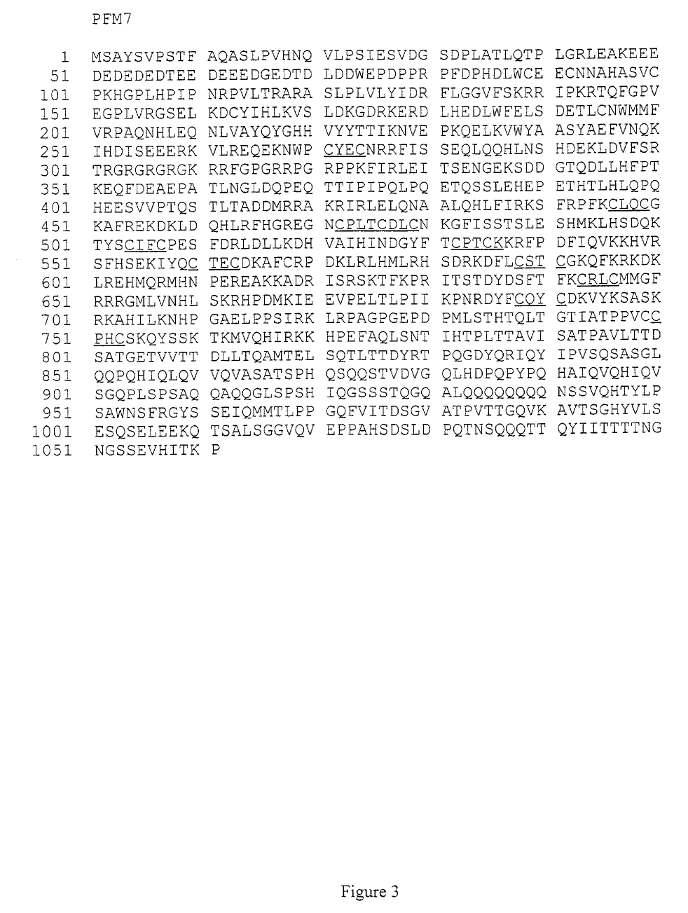

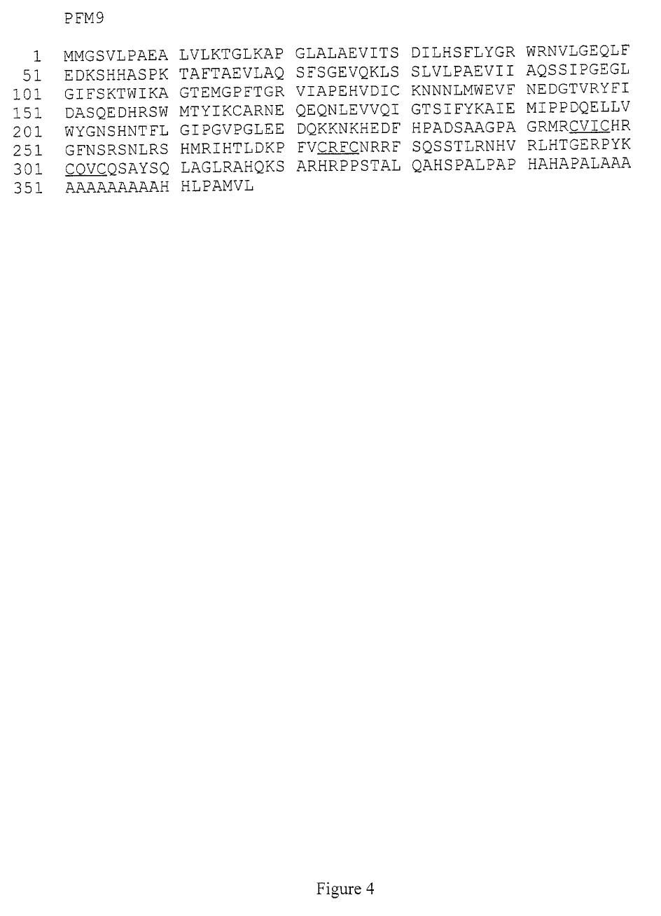

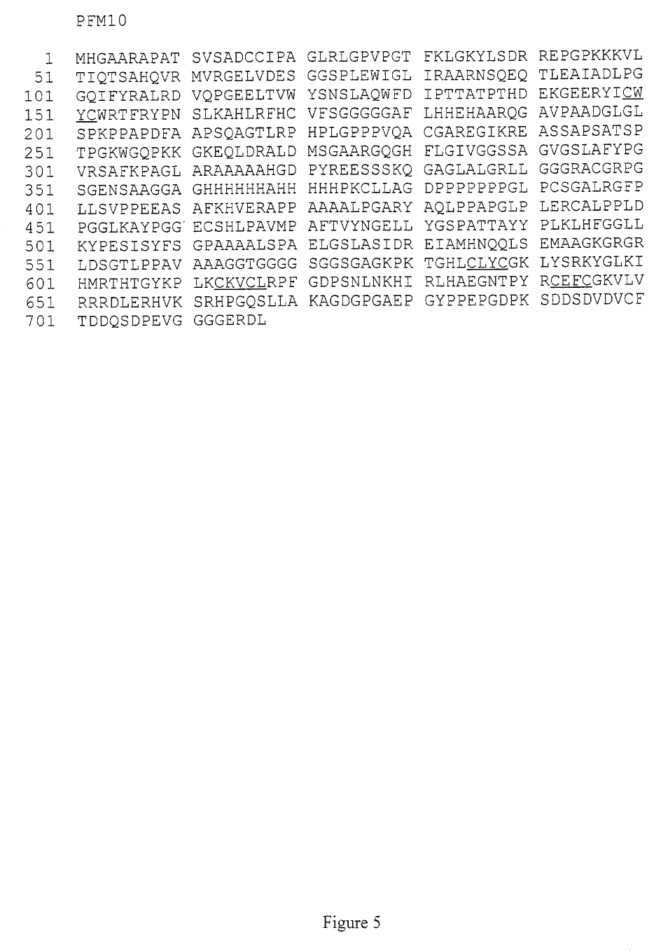

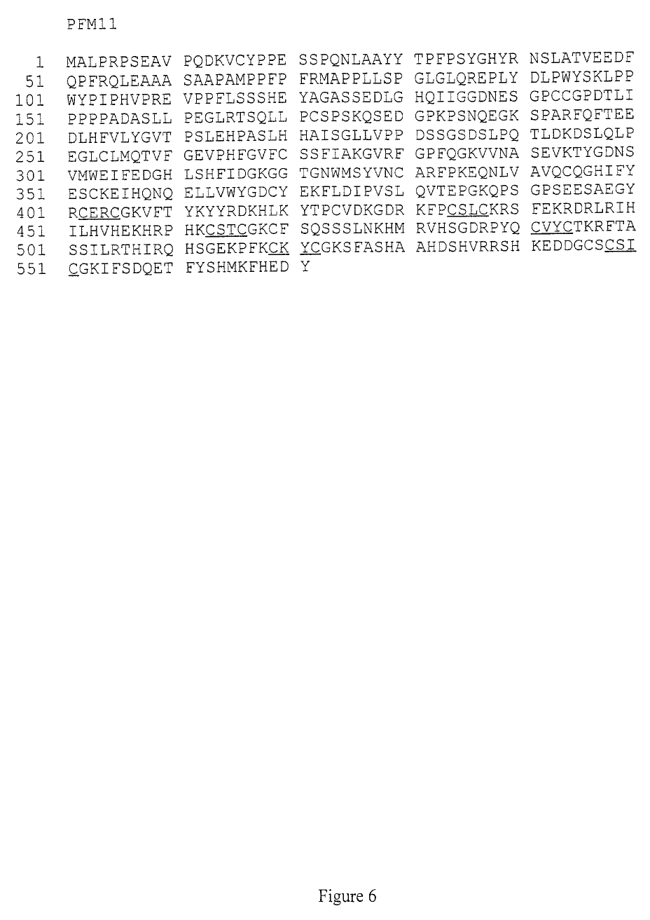

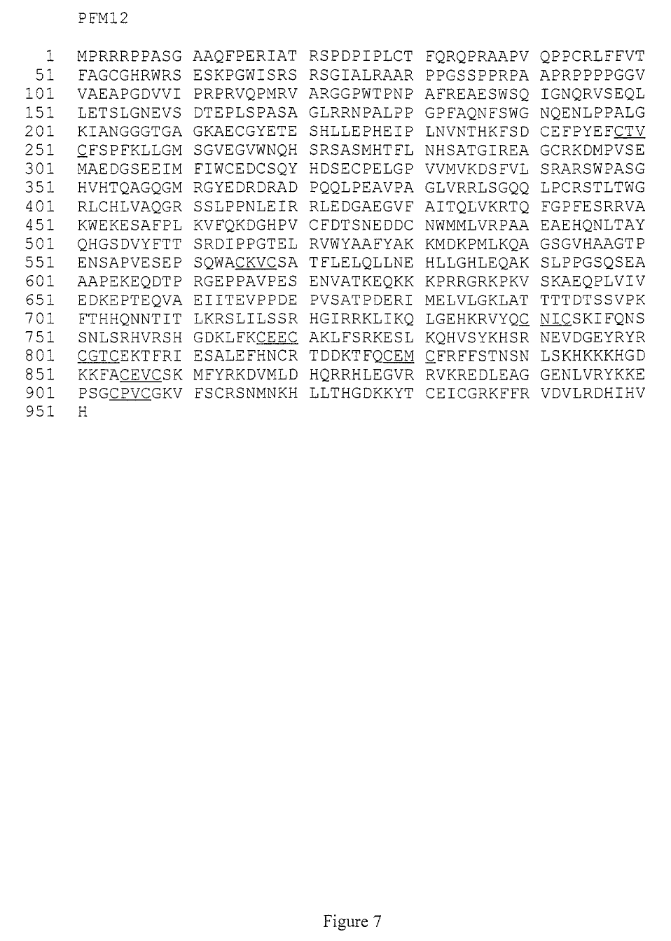

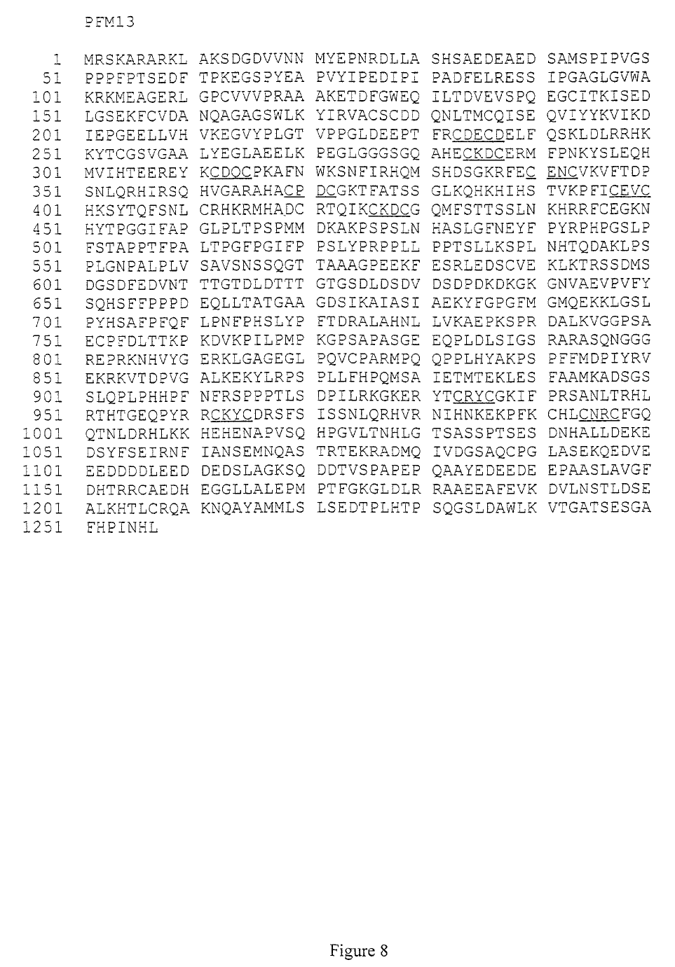

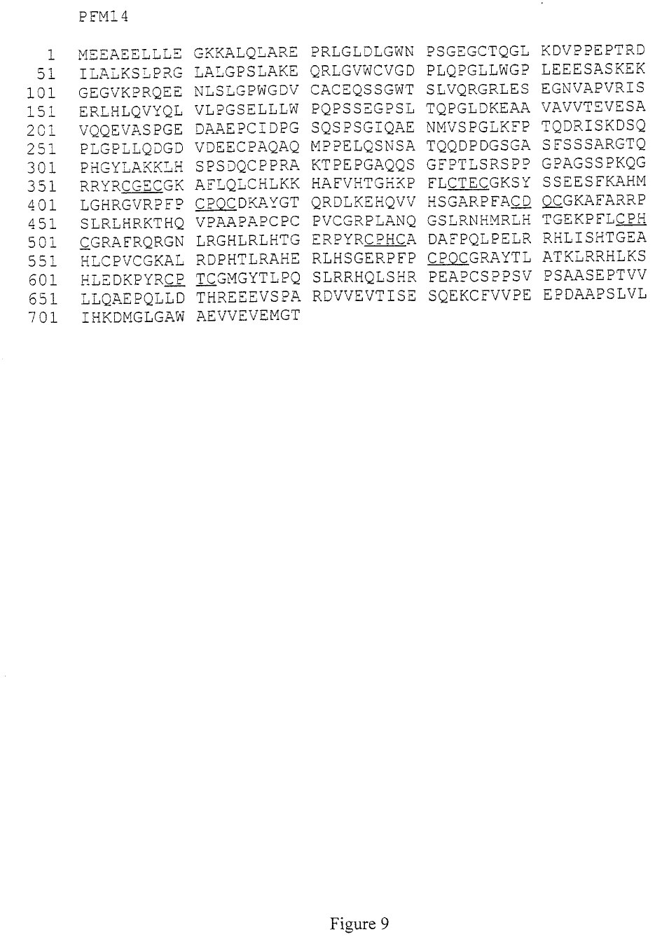

The zinc finger motifs within the PFM/SET nucleic acid molecules and polypeptides of the invention can be contained in one or more zinc finger domains. For example, the 10 zinc finger motifs of PFM7 are contained in a single zinc finger domain (amino acids 271-752) while the 10 zinc finger motifs of PFM13 are contained in two separate zinc finger domains (amino acids 149-169 and 585-682). The individual zinc finger motifs of each invention PFM/SET polypeptide are depicted in FIGS. 2 through 11, which show the amino acid sequences of PFM6, PFM7, PFM9, PFM10, PFM11, PFM12, PFM13, PMF14, and SET07, respectively. Zinc finger motifs are identified by underlining.

PFM/SET nucleic acid molecules and encoded polypeptides can additionally contain a variety of other motifs, including, for example, acidic motifs, PKZL domains and PRAZ domains, as described below for each PFM/SET nucleic acid molecule of the invention.

The PFM/SET nucleic acid molecules and polypeptides of the invention are designated PFM6, PFM7, PFM8, PFM9, PFM10, PFM11, PFM12, PFM13, PFM14 and SET07. The nucleotide sequences of these PFM nucleic acid molecules are set forth as SEQ ID NO:1 (PFM6); SEQ ID NO:3 (PFM7); SEQ ID NO:5 (PFM8); SEQ ID NO:7 (PFM9); SEQ ID NO:9 (PFM10); SEQ ID NO:11 (PFM11); SEQ ID NO:13 (PFM12); SEQ ID NO:15 (PFM13), SEQ ID NO:17 (PFM14) and SEQ ID NO:19 (SET07). The amino acid sequences of the encoded PFM polypeptides are set forth as SEQ ID NO:2 (PFM6); SEQ ID NO:4 (PFM7); SEQ ID NO:6 (PFM8); SEQ ID NO:8 (PFM9); SEQ ID NO:10 (PFM10); SEQ ID NO:12 (PFM11); SEQ ID NO:14 (PFM12); SEQ ID NO:16 (PFM13), SEQ ID NO:18 (PFM14) and SEQ ID NO:10 (SET07).

The amino acid boundaries of the PR and ZF domains of PFM6, PFM7, PFM8, PFM9, PFM10, PFM11, PFM12, PFM13 and PFM14 are provided in Table 2, below.

TABLE 2

| | PR domain | ZF domain |
|---|---|---|
| | amino acid | amino acid |
| PFM molecule | residues | residues |
| PFM6 | 1-160 | 335-690 |
| PFM7 | 116-258 | 271-753 |
| PFM8 | 128-165 | not present |
| PFM9 | 84-217 | 245-333 |
| PFM10 | 1-131 | 149-169, 585-682 |
| PFM11 | 252-292 | 402-566 |
| PFM12 | 414-539 | 248-277, 565-951 |
| PFM13 | 93-218 | 219-448, 915-1030 |
| PFM14 | 45-182 | 355-624 |

PFM6 additionally contains a domain of approximately 100 amino acids having about 35-40% identity to the KRAB-domain-containing zinc finger protein 133 (ZNF133). This domain is designated PKZL, for “PR and KRAB zinc finger protein-linked.” The PKZL domain of PFM6 corresponds to amino acids 211-310 of SEQ ID NO:2. The PKZL domain likely is important in mediating protein-protein interactions with cellular regulatory molecules.

PFM7, PFM8 and PFM12 additionally contain a PRAZ domain. PFM6 contains a partial PRAZ domain at the N-terminus. PRAZ stands for PR-domain Associated Zinc Finger motif. The signature sequence of the PRAZ domain is CX₂CX₇CX₂H/L. The PRAZ domain is typically located 5′ to the PR domain in PFM polypeptide sequences, and has thus far been identified only in PFM polypeptides. The PRAZ domain participates in the methyltransferase activity function of the PR domain. The PRAZ domain of PFM6 corresponds to amino acids 1-13 of SEQ ID NO:2. The PRAZ domain of PFM7 corresponds to amino acids 87-108 of SEQ ID NO:4. The PRAZ domain of PFM8 corresponds to amino acids 103-117 of SEQ ID NO:6. The PRAZ domain of PFM12 corresponds to amino acids 313-328 of SEQ ID NO:14.

The SET07 polypeptide of the invention contains a single SET domain. The SET domain of SET07 corresponds to amino acids 215-334 of SEQ ID NO:20.

The PFM and SET genes disclosed herein are localized to regions of human chromosomes predicted to harbor tumor suppressor genes, because deletion of these regions is closely associated with various human tumors. In particular, PFM6 localizes to chromosome band 5p14, a region commonly rearranged or gained in ovarian and breast cancer (Sonoda et al. Genes, Chromosomes & Cancer 20(4):320-8, (1997)).

PFM7 localizes to chromosome band 11q25, a region commonly deleted in breast, ovary, colon and oral cancer (Connolly et al., Cancer Research 59:2806-2809 (1999); Koreth et al., Oncogene 14:431-437 (1997); Launonen et al., Genecologic Oncoloqy 71:299-304 (1998); Uzawa et al., Intl. J. Cancer 67:510-4 (1996)). It has also been demonstrated that llq25 YAC clones can suppress tumor growth in vivo (Koreth et al., Oncogene 18:1157-64 (1999)).

PFM8 localizes to chromosome band 11p11. This region of chromosome 11 is commonly deleted in breast cancer (Nakata et al., European J. Cancer 34:417-21 (1998)).

PFM9 localizes to chromosome band chromosome 9q33-34.1, which is commonly deleted in ovarian, bladder, esophageal, and lung cancers (Devlin et al., British J. Cancer 73:420-423 (1996); Hornigold et al., Oncogene 18:2657-61 (1999); Simoneau et al., Oncogene 18:157-63 (1999)).

PFM10 localizes to chromosome band 6q16-21, a region commonly deleted in B-cell lymphoma, melanoma, and stomach cancers (Gaidano et al. Curr. Opin. Oncol. 5:778-784 (1993), Millikin et al. Cancer Res. 51:5449- 5453 (1991), Trent et al. Cytogenet. Cell Genet. 62:67- 87 (1990), and Queimado et al. Genes Chromosomes Cancer 14:28-34(1995)). This region also contains another PR-family member, the candidate tumor suppressor PRDIBF1/BLIMP1 (PRDM1) (Mock et al., Genomics 37:24-28 (1996)).

PFM11 localizes to chromosome band 8p12-21, a region commonly deleted in breast and prostate cancers (Van Alewijk et al., Genes, Chromosomes & Cancer 24:119-26 (1999); Verma et al., Cancer Investigation 17:441-7 (1999); Vocke et al., Cancer Research 56:2411-6 (1996)).

PFM12 localizes to chromosome band 21q22.3. This region is found to be deleted in human leukemia.

PFM13 localizes to chromosome band 1p36.23-33, a region deleted in more than a dozen different types of human cancers. This region also contains other PR-family members, the tumor suppressors MDS1-EVI1 and RIZ1. These observations are consistent with a role for these PFMs as tumor suppressors.

SET07 localizes to chromosome band 13q11-q13, in proximity to the retinoblastoma locus on 13q14. This region is commonly deleted in many cancers including breast cancers, bladder cancers, lung cancers and osteosarcomas.

PFM/SET nucleic acid molecules

The invention provides isolated PFM/SET nucleic acid molecules. The isolated PFM/SET nucleic acid molecules of the invention can be used in a variety of diagnostic and therapeutic applications. For example, as described in more detail below, the isolated PFM/SET nucleic acid molecules of the invention can be used as probes and primers to detect PFM and SET nucleic acid molecules in samples; as templates for the recombinant expression of PFM and SET polypeptides; in two-hybrid assays to identify cellular molecules that bind PFM and SET; and in vivo and ex vivo gene therapy applications to positively or negatively modulate cell proliferation.

In one embodiment, the invention provides an isolated PFM/SET nucleic acid molecule encoding a PFM/SET polypeptide selected from the group consisting of SEQ ID NOS:2, 4, 6, 8, 10, 12, 14, 16, 18 and 20. Modifications of these sequences that having at least 80% identity thereto are also provided. Such modifications can have at least 90%, 95% and 98% identity with a reference PFM/SET nucleotide sequence.

Exemplary isolated PFM/SET nucleic acid molecules provided by the invention are nucleic acid molecules having the sequence of SEQ ID NOS:1, 3, 5, 7, 8, 11, 13, 15, 17, or 19.

The invention also provides an isolated nucleic acid molecule containing a nucleotide sequence encoding a functional fragment of a PFM/SET polypeptide, said fragment comprising a PR, SET, PRAZ, or PKZL domain of a PFM/SET amino acid sequence selected from the group consisting of SEQ ID NOS:2, 4, 6, 8, 10, 12, 14, 16, 18 and 20.

The term “isolated,” in reference to a PFM/SET nucleic acid molecule is intended to mean that the molecule is substantially removed or separated from components with which it is naturally associated, or otherwise modified by the hand of man. Thus, the term “isolated PFM/SET nucleic acid molecule” excludes PFM/SET nucleic acid molecules as they exist in nature.

The term “nucleic acid molecule,” as used herein, refers to an oligonucleotide or polynucleotide of natural or synthetic origin. A nucleic acid molecule can be single- or double-stranded genomic DNA, cDNA or RNA, and can represent the sense strand, the antisense strand, or both.

Identity of any two nucleic acid sequences can be determined by those skilled in the art based, for example, on a BLAST 2.0 computer alignment. BLAST 2.0 searching is available at the National Institutes of Health web site (www.ncbi.nlm.nih.gov), and is described in Tatusova et al., FEMS Microbiol Lett. 174:247-250 (1999).

A “modification” of a reference nucleic acid sequence can include one or several nucleotide additions, deletions, or substitutions with respect to the recited sequence. Such modifications can correspond to variations that are made deliberately, or which occur as mutations during nucleic acid replication.

Exemplary “modifications” of the recited PFM/SET sequences include sequences that correspond to homologs of other species, such as primates, mouse, rat, rabbit, bovine, porcine, ovine, canine or feline species. The sequences of corresponding PFM/SET polypeptides of non-human species can be determined by methods known in the art, such as by PCR or by screening genomic, cDNA or expression libraries.

Furthermore, exemplary “modifications” of the recited PFM/SET nucleic acid or polypeptide can correspond to splice variant forms of recited PFM/SET sequences. Thus, for example, a modification of a PFM nucleic acid molecule of the invention can lack one or more of the exons that encode the A, B or C boxes of the PR domain. A, B, and C boxes of a PR domain can be determined by alignment of PR domain nucleotide sequences with known PR domain A, B, and C box sequences, or by comparing the sequence of a PFM/SET cDNA to the sequence of the corresponding genomic DNA. Exon usage by splice variants of PFM/SET nucleic acid molecules can be readily determined by those skilled in the art by comparing the sequence of the PFM/SET cDNA to the sequence of the corresponding PFM/SET genomic DNA.

Additionally, a “modification” of a reference sequence can include one or more non-native nucleotides, having, for example, modifications to the base, the sugar, or the phosphate portion, or having a modified phosphodiester linkage. Such modifications can be advantageous in increasing the stability of the nucleic acid molecule.

Furthermore, a “modification” of a reference sequence can include, for example, a detectable moiety, such as a radiolabel, a fluorochrome, a ferromagnetic substance, a luminescent tag or a detectable binding agent such as biotin. Such modifications can be advantageous in applications where detection of a PFM nucleic acid molecule is desired.

Nucleic acid molecules having nucleic acid sequences that encode modified polypeptides that are immunologically equivalent to the recited PFM amino acid sequences are also provided.

The term “isolated PFM/SET nucleic acid molecule” specifically excludes nucleic acid molecules consisting of certain nucleotide sequences, such as Expressed Sequence Tags (ESTs), Sequence Tagged Sites (STSs) and genomic fragments, deposited in public databases such as the nr, dbest, dbsts, gss and htgs databases, which are available for searching at http://www.ncbi.nlm.nih.gov/blast/blast.cgi?Jform=0, using the program BLASTN 2.0.9 [May 07, 1999] described by Altschul et al., Nucleic Acids Res. 25:3389-3402 (1997).

In particular, the term “PFM/SET nucleic acid molecule” specifically excludes nucleic acid molecules consisting of any of the nucleotide sequences having the GenBank (gb), EMBL (emb) or DDBJ (dbj) Accession numbers set forth in Table 3, below:

TABLE 3

| | |
|---|---|
| PFM nucleic | Accession Number of Excluded |
| acid | Sequence |

| | | |
|---|---|---|
| PFM6 | gb\|AC010432.6 | gb\|AF274348.1 |
| | gb\|AC025451.5 | emb\|AL137711 |
| | gb\|AF274347.1 | emb\|Z96314 |
| PFM7 | dbj\|AB033057 | gb\|BE962422.1 |
| | dbj\|AK000234 | gb\|BE994100.1 |
| | gb\|AA324539.1 | gb\|BF333514.1 |
| | gb\|AA331245.1 | gb\|BF693866.1 |
| | gb\|AA807222.1 | gb\|BG498843.1 |
| | gb\|AI348386.1 | gb\|G05930.1 |
| | gb\|AI610587.1 | gb\|G20316.1 |
| | gb\|AT699177.1 | gb\|N32595.1 |
| | gb\|AI907429.1 | gb\|R14616.1 |
| | gb\|AI917820.1 | gb\|R15777.1 |
| | gb\|AW027068.1 | gb\|R16283.1 |
| | gb\|AW131841.1 | gb\|R35195.1 |
| | gb\|AW274834.1 | gb\|R42665.1 |
| | gb\|AW978331.1 | gb\|R45605.1 |
| | gb\|AW992560.1 | gb\|R50855.1 |
| | gb\|BE304522.1 | gb\|T16683.1 |
| | gb\|BE617458.1 | |
| PFM8 | gb\|AA468023.1 | gb\|BE884008.1 |
| | gb\|AA468074.1 | gb\|BE956829.1 |
| | gb\|AC013602.4 | gb\|BE980340.1 |
| | gb\|AW968153.1 | gb\|D59353.1 |
| | gb\|BE648497.1 | |
| PFM9 | gb\|AW529888.1 | gb\|BE096442.1 |
| | gb\|AW532948.1 | gb\|BE096447.1 |
| | gb\|BE096110.1 | gb\|BE294489.1 |
| | gb\|BE096155.1 | gi\|13274746 |
| PFM10 | emb\|AL137784.14 | emb\|AL035087.20 |
| PFM11 | dbj\|AK022595.1 | gb\|BF770200 |
| | dbj\|AU124563 | gb\|G51347.1 |
| | dbj\|AU148392 | gi\|13375633 |
| | emb\|Z65361.1 | |
| PFM12 | djb\|AP001618.1 | gb\|AW501914.1 |
| | djb\|AP001619.1 | gb\|AW503893.1 |
| | djb\|AP001745.1 | gb\|AW968839.1 |
| | gb\|AA279563.1 | gb\|BF904312.1 |
| | gb\|AA490433.1 | |
| PFM13 | dbj\|AB051462.1 | gb\|BG086572.1 |
| | dbj\|AK017846.1 | gi\|13628859 |
| | gb\|BF982577.1 | |
| PFM14 | dbj\|AU130916 | gb\|BE793683.1 |
| | emb\|AL535257 | gb\|BE798564.1 |
| | emb\|AL565845 | gb\|BE870276.1 |
| | gb\|AA635466.1 | gb\|BE883835.1 |
| | gb\|AA639997.1 | gb\|BF061011.1 |
| | gb\|AA759024.1 | gb\|BF182733.1 |
| | gb\|AI092401.1 | gb\|BF346948.1 |
| | gb\|AI242496.1 | gb\|BF529537.1 |
| | gb\|AI357201.1 | gb\|BF685622.1 |
| | gb\|AI816535.1 | gb\|BF814715.1 |
| | gb\|AU128198.1 | gb\|BG177268.1 |
| | gb\|AU147298.1 | gb\|BG470196.1 |
| | gb\|AU152780.1 | gb\|BG481345.1 |
| | gb\|AW129728.1 | gb\|BG678255.1 |
| | gb\|AW157409.1 | gb\|BG745296.1 |
| | gb\|AW245524.1 | gb\|BG746115.1 |
| | gb\|AW245967.1 | gb\|BG753045.1 |
| | gb\|AW246726.1 | gb\|BG753749.1 |
| | gb\|AW273736.1 | gb\|BG822286.1 |
| | gb\|BE244872.1 | gb\|BG914332.1 |
| | gb\|BE246083.1 | gb\|BI116711.1 |
| | gb\|BE247252.1 | gb\|BI117664.1 |
| | gb\|BE732157.1 | gb\|H63042.1 |
| | gb\|BE744525.1 | gb\|H85444.1 |
| | gb\|BE791132.1 | |
| | gb\|AI887341.1 | gb\|BE897305.1 |
| | gb\|AW163472.1 | gb\|H85725.1 |
| | gb\|BE048089.1 | gb\|R37802.1 |
| | gb\|BE266801.1 | |
| | gb\|BE867579.1 | |
| SET07 | dbj\|AV752467 | gb\|BE018920.1 |
| | emb\|AL120271.1 | gb\|BE074968.1 |
| | emb\|AL548156.1 | gb\|BE074969.1 |
| | emb\|AL555671 | gb\|BE074974.1 |
| | emb\|AL578116 | gb\|BE305526.1 |
| | gb\|912371.1 | gb\|BE536337.1 |
| | gb\|AA085455.1 | gb\|BE797607.1 |
| | gb\|AA509936.1 | gb\|BE896201.1 |
| | 9b\|AA936703.1 | gb\|BF133687.1 |
| | gb\|AI002388.1 | gb\|BF161282.1 |
| | gb\|AI383837.1 | gb\|BF245288.1 |
| | gb\|AI879547.1 | gb\|BF309152.1 |
| | gb\|AV705547.1 | gb\|BG196219.1 |
| | gb\|AW430084.1 | gb\|BG311741.1 |
| | gb\|AW986692.1 | gb\|BG389847.1 |
| | gb\|BE014950.1 | gb\|BG922564.1 |
| | | gb\|BI021983.1 |
| | | gb\|W72745.1 |

The invention also provides isolated PFM Oligonucleotides containing at least 17 contiguous nucleotides of PFM6, PFM7, PFM8, PFM9, PFM10, PFM11, PFM12, PFM13, PFM14 or SET07. As used herein, the term “oligonucleotide” refers to a nucleic acid molecule that includes at least 17 contiguous nucleotides from the reference nucleotide sequence, can include at least 16, 17, 18, 19, 20 or at least 25 contiguous nucleotides, and often includes at least 30, 40, 50, 60, 70, 80, 90, 100, 125, 150, 175, 200, 225, 250, 275, 300, 325, 350, 450, 550, 650, 750, 850, 950 or more contiguous nucleotides from the reference nucleotide sequence. Therefore, an oligonucleotide can include the entire nucleotide sequence encoding a PFM/SET polypeptide. An oligonucleotide can further contain an exogenous nucleotide sequence. Such exogenous nucleotide sequence includes, for example, a nucleotide sequence that facilitates identification or purification of the oligonucleotide, a nucleotide sequence that facilitates cloning, such as a sequence containing a restriction endonuclease recognition site, or any other nucleotide sequence not contained in a naturally occurring reference PFM/SET nucleotide sequence. Excluded from the nucleotide sequence of an invention PFM/SET oligonucleotide is non-coding sequence adjacent to a naturally occurring PFM/SET nucleotide sequence, such as nucleotide sequence contained upstream or downstream of a naturally occurring PFM/SET nucleotide sequence, or portion thereof, in genomic or cDNA.

The PFM/SET oligonucleotides of the invention contain at least 17 contiguous nucleotides from the reference PFM or SET nucleotide sequence and are able to hybridize to PFM or SET nucleotide sequences under moderately stringent hybridization conditions. Therefore, PFM/SET oligonucleotides can be advantageously used, for example, as probes to detect PFM or SET DNA or RNA in a sample, and to detect splice variants thereof that contain or lack particular domains; as sequencing or PCR primers; as antisense reagents to block transcription of PFM or SET RNA in cells; or in other applications known to those skilled in the art in which hybridization to a PFM or SET is desirable.

Oligonucleotides containing at least 17 contiguous PFM/SET nucleotides are able to specifically hybridize with a PFM or SET nucleic acid molecule. Specific hybridization refers to the ability of a nucleic acid molecule to hybridize, under moderately stringent conditions as described above, to the reference PFM or SET nucleic acid molecule, without hybridization under the same conditions with nucleic acid molecules that are not PFMs or SETs, respectively, such as actin cDNA.

Moderately stringent hybridization conditions refers to hybridization conditions that permit a nucleic acid molecule to bind a nucleic acid that has substantial identity to the recited sequence. Moderately stringent conditions are conditions equivalent to hybridization of filter-bound nucleic acid in 50% formamide, 5×Denhart's solution, 5×SSPE, 0.2% SDS at 42° C., followed by washing in 0.2×SSPE, 0.2% SDS, at 50°. In contrast, “highly stringent conditions” are conditions equivalent to hybridization of filter-bound nucleic acid in 50% formamide, 5×Denhart's solution, 5×SSPE, 0.2% SDS at 42° C., followed by washing in 0.2×SSPE, 0.2% SDS, at 65°. Other suitable moderately stringent and highly stringent hybridization buffers and conditions are well known to those of skill in the art and are described, for example, in Sambrook et al., Molecular Cloning: A Laboratory Manual, Cold Spring Harbor Laboratory, New York (1992) and in Ansubel et al., Current Protocols in Molecular Biology, John Wiley and Sons, Baltimore, Md. (1998).

In general, a nucleic acid molecule that hybridizes to a recited sequence under moderately stringent conditions will have greater than about 60% identity, such as greater than about 70% identity, preferably greater than about 80% identity to the reference sequence over the length of the two sequences being compared. A nucleic acid molecule that hybridizes to a recited sequence under highly stringent conditions will generally have greater than about 90% identity, including greater than about 95% and 98% identity, to the reference sequence over the length of the two sequences being compared.

In one embodiment, the invention provides a primer pair for detecting a PFM or SET nucleic acid. The primer pair contains two PFM/SET oligonucleotides. The primer pair can be used, for example, to amplify PFM/SET DNA by RT-PCR or PCR.

The isolated PFM/SET nucleic acid molecules and oligonucleotides of the invention can be produced or isolated by methods known in the art. The method chosen will depend, for example, on the type of nucleic acid molecule one intends to isolate. Those skilled in the art, based on knowledge of the nucleotide sequences disclosed herein, can readily isolate PFM/SET nucleic acid molecules as genomic DNA, or desired introns, exons or regulatory sequences therefrom; as full-length cDNA or desired fragments therefrom; or as full-length mRNA or desired fragments therefrom, by methods known in the art.

One useful method for producing an isolated PFM/SET nucleic acid molecule of the invention involves amplification of the nucleic acid molecule using the polymerase chain reaction (PCR) and PFM- or SET-specific primers and, optionally, purification of the resulting product by gel electrophoresis. Either PCR or reverse-transcription PCR (RT-PCR) can be used to produce a PFM nucleic acid molecule having any desired nucleotide boundaries. Desired modifications to the nucleic acid sequence can also be introduced by choosing an appropriate primer with one or more additions, deletions or substitutions. Such nucleic acid molecules can be amplified exponentially starting from as little as a single gene or mRNA copy, from any cell, tissue or species of interest.

A further method of producing an isolated PFM/SET nucleic acid molecule of the invention is by screening a library, such as a genomic library, cDNA library or expression library, with a detectable agent. Such libraries are commercially available or can be produced from any desired tissue, cell, or species of interest using methods known in the art. For example, a cDNA or genomic library can be screened by hybridization with a detectably labeled nucleic acid molecule having a nucleotide sequence disclosed herein. Additionally, an expression library can be screened with an antibody raised against a polypeptide corresponding to the coding sequence of a PFM/SET nucleic acid disclosed herein. The library clones containing PFM molecules of the invention can be isolated from other clones by methods known in the art and, if desired, fragments therefrom can be isolated by restriction enzyme digestion and gel electrophoresis.

Furthermore, isolated PFM/SET nucleic acid molecules and oligonucleotides of the invention can be produced by synthetic means. For example, a single strand of a nucleic acid molecule can be chemically synthesized in one piece, or in several pieces, by automated synthesis methods known in the art. The complementary strand can likewise be synthesized in one or more pieces, and a double-stranded molecule made by annealing the complementary strands. Direct synthesis is particularly advantageous for producing relatively short molecules, such as oligonucleotide probes and primers, and nucleic acid molecules containing modified nucleotides or linkages.

The invention also provides a vector containing an isolated PFM/SET nucleic acid molecule. The vectors of the invention are useful for subcloning and amplifying an isolated PFM/SET nucleic acid molecule, and for recombinantly expressing a PFM/SET polypeptide. A vector of the invention can include a variety of elements useful for cloning and/or expression of PFM/SET nucleic acid molecules, such as enhancer sequences and promoter sequences from a viral, bacterial or mammalian gene, which provide for constitutive, inducible or cell-specific RNA transcription; transcription termination and RNA processing signals, including polyadenylation signals, which provide for stability of a transcribed mRNA sequence; an origin of replication, which allows for proper episomal replication; selectable marker genes, such as a neomycin or hygromycin resistance gene, useful for selecting stable or transient transfectants in mammalian cells, or an ampicillin resistance gene, useful for selecting transformants in prokaryotic cells; and versatile multiple cloning sites for inserting nucleic acid molecules of interest.

Cloning vectors of the invention include, for example, viral vectors such as a bacteriophage, a baculovirus or a retrovirus; cosmids or plasmids; and, particularly for cloning large nucleic acid molecules, bacterial artificial chromosome vectors (BACs) and yeast artificial chromosome vectors (YACs). Such vectors are commercially available, and their uses are well known in the art.

If it is desired to express PFM/SET RNA transcripts or polypeptides, a PFM/SET nucleic acid molecule can be inserted into an expression vector such that it is operatively linked to a promoter of RNA transcription. The term “operatively linked,” as used herein, is intended to mean that the nucleic acid molecule is positioned with respect to a PFM/SET promoter, or heterologous promoter, in such a manner that the promoter will direct the transcription of RNA using the nucleic acid molecule as a template. Methods for operatively linking a nucleic acid to a desired promoter are well known in the art and include, for example, cloning the nucleic acid into a vector containing the desired promoter, or appending the promoter to a nucleic acid sequence using PCR. Thus, an expression vector containing a PFM/SET nucleic acid molecule operatively linked to a promoter of RNA transcription can be used to express PFM/SET transcripts and polypeptides in a desired host cell, or in an in vitro system, such as an extract or lysate that supports transcription and translation. Contemplated expression vectors include vectors containing regulatory sequences known in the art to provide for expression in bacterial cells, yeast cells, insect cells, mammalian cells and other vertebrate cells.

A variety of expression vectors are commercially available, and can be further modified, if desired, to include appropriate regulatory elements to provide for the desired level of expression or replication in the host cell. For example, appropriate promoter and enhancer elements can be chosen to provide for constitutive, inducible or cell type-specific expression. Useful constitutive promoter and enhancer elements for expression of PFM/SET in mammalian cells include, for example, RSV, CMV, SV40 and IgH elements. An exemplary inducible expression element is a steroid response element, while an exemplary cell-specific expression element is a prostate specific antigen (PSA) regulatory sequence. Other constitutive, inducible and cell type-specific regulatory elements are well known in the art.

Exemplary host cells that can be used to express recombinant PFM/SET molecules include mammalian primary cells; established mammalian cell lines, such as COS, CHO, HeLa, NIH3T3, HEK 293-T and PC12 cells; amphibian cells, such as Xenopus embryos and oocytes; and other vertebrate cells. Exemplary host cells also include insect cells (e.g. Drosophila), yeast cells (e.g. S. cerevisiae, S. pombe, or Pichia pastoris) and prokaryotic cells (e.g. E. coli).

Methods for introducing a cloning or expression vector into a host cell are well known in the art and include, for example, various methods of transfection such as calcium phosphate, DEAE-dextran and lipofection methods, viral transduction, electroporation and microinjection. Host cells expressing PFM or SET nucleic acid molecules can be used, for example, as a source to isolate recombinantly expressed PFM or SET polypeptides, to identify and isolate molecules that regulate or interact with PFM/SET nucleic acids and polypeptides, or to screen for compounds that enhance or inhibit the activity of a PFM/SET molecule of the invention, as described further below.

The methods of isolating, cloning and expressing nucleic acid molecules of the invention referred to herein are routine in the art and are described in detail, for example, in Sambrook et al., Molecular Cloning: A Laboratory Manual, Cold Spring Harbor Laboratory, New York (1992) and in Ansubel et al., Current Protocols in Molecular Biology, John Wiley and Sons, Baltimore, Md. (1998), which are incorporated herein by reference.

PFM/SET polypeptides

The invention also provides isolated PFM/SET polypeptides. As disclosed herein, PFM and SET domains are similar in both amino acid sequence and in biological function. As such, a PFM and SET domains are members of a class of domains, referred to herein as PFM/SET domains. The isolated PFM/SET nucleic acid molecules and polypeptides of the invention contain either a PFM or SET domain. The isolated PFM and SET polypeptides of the invention can be used in a variety of diagnostic and therapeutic applications. For example, as described in more detail below, the isolated PFM/SET polypeptides can be used to generate antibodies that can be used as reagents to detect PFM or SET mRNA or polypeptide expression in a sample, or in screening methods to identify compounds and cellular molecules that bind PFM or SET polypeptides and modulate cell proliferation.

In one embodiment, the invention provides an isolated polypeptide, containing a PFM/SET amino acid sequence selected from the group consisting of SEQ ID NOS:2, 4, 6, 8, 10, 12, 14, 16, 18 and 20. Also provided are PFM/SET polypeptides that have at least 80% identity thereto. Further provided are isolated PFM/SET polypeptides having at least about 90%, 95% and 98% identity with a reference PFM/SET polypeptide amino acid sequence.

Exemplary isolated polypeptides provided by the invention are PFM/SET polypeptides having the sequence of SEQ ID NOS:2, 4, 6, 8, 10, 12, 14, 16, 18 or 20.

The term “isolated,” in reference to a PFM/SET polypeptide of the invention, is intended to mean that the molecule is substantially removed or separated from components with which it is naturally associated, or otherwise modified by the hand of man. Thus, the term “isolated PFM/SET polypeptide” excludes PFM and SET polypeptides as they exist in nature.

Isolated PFM/SET polypeptides that have at least about 90%, 95% and 98% identity with a reference PFM/SET polypeptide amino acid sequence can further be immunologically equivalent to a reference PFM/SET polypeptide. An immunologically equivalent PFM/SET polypeptide can be recognized by an antibody that also specifically binds to the reference PFM/SET sequence. Specific binding refers to high affinity binding of an antibody to the subject polypeptide, and binding with substantially lower affinity to an unrelated polypeptide, such as bovine serum albumin. High affinity binding includes binding with a dissociation constant (Kd) of less than about 10⁻⁶ M, preferably less than about 10⁻⁷ M, such as less than about 10⁻⁸ M. Methods of determining binding affinity are well known in the art and are described, for example, in Harlow and Lane, Antibodies: A Laboratory Manual, Cold Spring Harbor Laboratory, New York (1989).

Those skilled in the art understand that two polypeptides with a high percentage of identity over the entire sequence, or over a substantial portion of the sequence, are more likely to exhibit similar biological activities than two molecules with the same percentage identity over a shorter portion of the sequence. Furthermore, two polypeptides that fold into common epitope structures are also more likely to exhibit similar biological activities than two molecules that do not share a common three-dimensional structure. Accordingly, an amino acid sequence that is “immunologically equivalent” to a PFM/SET polypeptide can further be “functionally equivalent” to a PFM/SET polypeptide. An exemplary “functionally equivalent” PFM/SET polypeptide is a PFM/SET “functional fragment.”

The invention further provides a modification of a PFM/SET polypeptide. As used herein, a “modification” of a PFM/SET polypeptide of a reference amino acid sequence can include one or more additions, deletions or substitutions with respect to the reference sequence. In particular, a modification can include a conservative substitution, such as substitution of an apolar amino acid with another apolar amino acid (such as replacement of leucine with isoleucine), or substitution of a charged amino acid with a similarly charged amino acid (such as replacement of a glutamic acid with an aspartic acid). A modification can also include a nonconservative change, wherein a substituted amino acid has different but sufficiently similar structural or chemical properties that permits such a substitution without adversely affecting the desired immunological or biological activity.

A “modification” of a reference amino acid sequence that is “immunologically equivalent” or “biologically equivalent” to the reference amino acid sequence can also be a chemical or enzymatic modification, including but not limited to replacement of hydrogen by an alkyl, acyl, or amino group; esterification of a carboxyl group with a suitable alkyl or aryl moiety; alkylation of a hydroxyl group to form an ether derivative; phosphorylation or dephosphorylation of a serine, threonine or tyrosine residue; or−or O-linked glycosylation.

Exemplary “modifications” of the recited PFM or SET sequences include sequences that correspond to homologs of other species, such as primates, mouse, rat, rabbit, bovine, porcine, ovine, canine or feline species. Furthermore, exemplary “modifications” of the recited PFM or SET sequences can correspond to splice variant forms, or internal translation products, of a recited PFM or SET sequence. Thus, for example, a modification of a PFM polypeptide of the invention can lack one or more of the A, B or C boxes of the PR domain.

Those skilled in the art can determine appropriate amino acid modifications for a given application. For example, a modification can serve to increase the stability, bioavailability, bioactiviy or immunogenicity of the polypeptide, or to facilitate its purification. Thus, introduction of a D-amino acid or an amino acid analog for its corresponding L-amino acid, or deletion of a lysine residue, can stabilize a polypeptide and reduce degradation. Addition of tag sequences, such as epitope tags, histidine tags, glutathione-S-transferase (GST) and the like, or addition of sorting sequences, can facilitate purification of a recombinant polypeptide. Addition of carrier sequences, such as keyhole limpet hemocyanin, can enhance recognition of the polypeptide by the immune system. Depending on the modification and the source of the polypeptide, the modification can be introduced into the polypeptide, or into the encoding nucleic acid sequence.

Computer programs known in the art can provide guidance in determining which amino acid residues can be modified as indicated above without abolishing the immunological activity or a desired biological activity of a PFM/SET polypeptide (see, for example, Eroshkin et al., Comput. Appl. Biosci. 9:491-497 (1993)). Additionally, guidance in modifying amino acid sequences while retaining functional activity is provided by aligning homologous PFM/SET polypeptides from various species. Those skilled in the art understand that evolutionarily conserved amino acid residues and domains are more likely to be important for maintaining biological activity than less well-conserved residues and domains.

In yet another embodiment, the invention provides an isolated PFM/SET peptide, having at least 8 contiguous amino acids of SEQ ID NOS:2, 4, 6, 8, 10, 12, 14, 16, 18 or 20. As used herein, the term “PFM/SET peptide” refers to a peptide having at least 8 contiguous amino acids of PFM/SET amino acid sequence. The amino acid length of functional fragments, peptides or polypeptide analogs of the present invention can range from about 8 amino acids up to the full-length protein sequence of an invention PR/SET polypeptide.

In certain embodiments, the amino acid lengths include, for example, at least about 10 amino acids, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 125, at least about 150, at least about 175, at least about 200, at least about 250 or more amino acids in length up to the full-length PFM/SET polypeptide sequence. A peptide of at least about 8 amino acids can be used, for example, as an immunogen to raise antibodies specific for an invention PFM/SET polypeptide.

A peptide of such size contains at least one epitope specific to PFM/SET, and can thus be used as an immunogen to produce PFM/SET-specific antibodies, or as an antigen to purify PFM/SET antibodies. PFM/SET peptides that are likely to be antigenic or immunogenic can be predicted using methods and algorithms known in the art and described, for example, by Irnaten et al., Protein Eng. 11:949-955 (1998), and Savoie et al., Pac. Symp. Biocomput. 1999:182-189 (1999). Immunogenicity of the PFM or SET peptides of the invention can be determined by methods known in the art, such as assay of a delayed-type hypersensitivity response in an animal sensitized to a PFM/SET polypeptide, or by elicitation of PFM/SET specific antibodies. Likewise, antigenicity of the PFM/SET peptides of the invention can be determined by methods known in the art, such as by ELISA analysis, as described, for example, in Harlow and Lane, supra (1988).

As disclosed herein, the PFM/SET polypeptides of the invention share several biological activities. The biological activities of PFM/SET polypeptides include, for example, growth modulating activity, regulation of chromatin-mediated gene expression, specific binding to cellular proteins, specific binding to DNA, methyltransferase activity and other biological activities described herein. The biological activities of a PFM/SET polypeptide can be mediated by a domain within a full length PFM/SET polypeptide.

Therefore, the invention provides a PFM/SET polypeptide that is a functional fragment of a PFM/SET polypeptide, said fragment comprising a PR, SET, PRAZ, or PKZL domain of a PFM/SET amino acid sequence selected from the group consisting of SEQ ID NOS:2, 4, 6, 8, 10, 12, 14, 16, 18 and 20.

As used herein, the term “functional fragment” is intended to mean a portion of a PFM/SET polypeptide that has one or more of the biological activities characteristic of the reference polypeptide. As used herein, the term “fragment” refers to any truncated form, either carboxy-terminal, amino-terminal, or both, of the reference PFM/SET polypeptide. A functional fragment has an amino acid length required to maintain a portion of the biological activities characteristic of the reference PFM/SET polypeptide, from a few amino acids to a one amino acid deletion of the full length PFM/SET amino acid sequence.

In certain embodiments, the amino acid lengths of a functional fragment include, for example, at least about 10 amino acids, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 125, at least about 150, at least about 175, at least about 200, at least about 250 or more amino acids in length up to a one amino acid deletion of a full-length PFM/SET polypeptide sequence. The functional fragments can be contiguous amino acid sequences of an invention polypeptide, including contiguous amino acid sequences of SEQ ID NOS:2, 4, 6, 8, 10, 12, 14, 16, 18 and 20.

A functional fragment of a PFM/SET polypeptide can contain exogenous amino acid sequence that is not contained in a naturally occurring PFM/SET polypeptide. Exemplary exogenous amino acid sequences are tags that facilitate identification or purification of a PFM/SET polypeptide, such as histidine tags, glutathione-S transferase tags, FLAG tags and myc tags.

For example, a functional fragment of an invention polypeptide can contain or consist of one or more of the following: a PR domain, a SET domain, a zinc finger domain, a PKZL domain, a PRAZ domain or an acidic region. In a specific example, a fragment of a PFM/SET polypeptide can contain a zinc finger domain and PRAZ domain, but lack a functional PR domain. Such a fragment will maintain a portion of the naturally occurring biological activities of the PFM/SET polypeptide, but not all such activities. For example, such a PR domain-minus polypeptide would lack methyltransferase activity. The resultant fragment will therefore have an activity different than the reference PFM/SET polypeptide. In another example, the PFM/SET polypeptide might have only the PR domain, allowing it to have methyltransferase activity. The activity of the fragment can be, for example, “dominant-negative.” A dominant-negative activity will allow the fragment to reduce or inactivate the activity of one or more isoforms of a naturally occurring PFM/SET polypeptide. Another functional fragment can include a PRAZ domain, or any of the domains disclosed herein.

Another biological activity of a PFM/SET polypeptide is the ability to act as an immunogen for the production of antibodies, or other antigen binding molecules, that bind specifically to an invention PFM/SET polypeptide.

The PFM/SET peptides of the invention can also be used in screening methods to identify PFM/SET-binding cellular molecules and modulatory compounds, as described further below.

The isolated PFM/SET polypeptides and peptides of the invention can be prepared by methods known in the art, including biochemical, recombinant and synthetic methods. For example, PFM and SET polypeptides can be purified by routine biochemical methods from a cell or tissue source that expresses abundant amounts of the corresponding transcript or polypeptide. The diagnostic methods disclosed herein can be adapted for determining which cells and tissues, and which subcellular fractions therefrom, are appropriate starting materials. Biochemical purification can include, for example, steps such as solubilization of the appropriate tissue or cells, isolation of desired subcellular fractions, size or affinity chromatography, electrophoresis, and immunoaffinity procedures. The methods and conditions for biochemical purification of a polypeptide of the invention can be chosen by those skilled in the art, and purification monitored, for example, by an ELISA assay or a functional assay, such as a DNA-binding or protein-binding assay.

A PFM/SET polypeptide, functional fragment or peptide having any desired boundaries, and a polypeptide having a modification to the native PFM or SET amino acid sequences, can also be produced by recombinant methods. Recombinant methods involve expressing a nucleic acid molecule encoding the desired polypeptide or fragment in a host cell or cell extract, and isolating the recombinant polypeptide or fragment, such as by routine biochemical purification methods described above. To facilitate identification and purification of the recombinant polypeptide, it is often desirable to insert or add, in-frame with the coding sequence, nucleic acid sequences that encode epitope tags, polyhistidine tags, glutathione-S-transferase (GST) domains, and similar affinity binding sequences, or sequences that direct expression of the polypeptide in the periplasm or direct secretion. Methods for producing and expressing recombinant polypeptides in vitro and in prokaryotic and eukaryotic host cells are well known in the art.

Thus, the invention provides a method of isolating a PFM/SET polypeptide, by growing a host cell containing an expression vector encoding a PFM or SET polypeptide, under conditions appropriate for expression of the encoded PFM/SET polypeptide, and isolating the PFM/SET polypeptide.

The PFM/SET polypeptide fragments and peptides of the invention can also be produced, for example, by enzymatic or chemical cleavage of the full-length polypeptide. Methods for enzymatic and chemical cleavage and for purification of the resultant peptide fragments are well known in the art (see, for example, Deutscher, Methods in Enzymology, Vol. 182, “Guide to Protein Purification,” San Diego: Academic Press, Inc. (1990), which is incorporated herein by reference).

Furthermore, PFM/SET polypeptides and peptides can be produced by chemical synthesis. If desired, such as to optimize their functional activity, stability or bioavailability, such molecules can be modified to include D-stereoisomers, non-naturally occurring amino acids, and amino acid analogs and mimetics. Examples of modified amino acids and their uses are presented in Sawyer, Peptide Based Drug Design, ACS, Washington (1995) and Gross and Meienhofer, The Peptides: Analysis, Synthesis, Biology, Academic Press, Inc., New York (1983), both of which are incorporated herein by reference.

PFM/SET antibodies

The invention also provides an antibody or antigen binding fragment thereof which specifically binds a PFM/SET polypeptide. Such antibodies can be used, for example, to affinity purify a PFM/SET polypeptide from a cell or tissue source, or in therapeutic and diagnostic applications described below.

An “antigen binding fragment” of an antibody of the invention includes, for example, individual heavy or light chains and fragments thereof, such as VL, VH and Fd; monovalent fragments, such as Fv, Fab, and Fab′; bivalent fragments such as F(ab′)₂; single chain Fv (scFv); and Fc fragments. Antigen binding fragments include, for example, fragments produced by protease digestion or reduction of an antibody, as well as fragments produced by recombinant DNA methods known to those skilled in the art.

The antibodies of the invention can be produced by any method known in the art. For example, a PFM or SET polypeptide or immunogenic peptide of the invention, or a nucleic acid expressing such a polypeptide, can be administered to an animal, using standard methods, and polyclonal antibodies isolated therefrom. Such polypeptides of peptides, if desired, can be conjugated to a carrier, such as KLH, serum albumin, tetanus toxoid and the like, using standard linking techniques, to increase their immunogenicity. Additionally, such peptides can be formulated together with an adjuvant known in the art, such as Freund's complete or incomplete adjuvant. The antibodies so generated can be used in the form of serum isolated from an immunized animal, or the antibody can be affinity purified from the serum using PFM/SET peptides or polypeptides.

Additionally, the PFM/SET antibodies of the invention can be monoclonal antibodies produced by a hybridoma cell line, by chemical synthesis, or by recombinant methods. Modified antibodies, such as chimeric antibodies, humanized antibodies and CDR-grafted or bifunctional antibodies, can also be produced by methods well known to those skilled in the art.

Methods of preparing and using antibodies and antigen-binding fragments, including detectably labeled antibodies, are described, for example, in Harlow and Lane, Antibodies: A Laboratory Manual, Cold Spring Harbor Laboratory, New York (1989); in Day, E. D., Advanced Immunochemistry, Second Ed., Wiley-Liss, Inc., New York, N.Y. (1990); and in Borrebaeck (Ed.), Antibody Engineerinq, Second Ed., Oxford University Press, New York (1995), which are incorporated herein by reference.

PFM/SET modulatory compounds

The invention also provides methods of identifying cellular and non-cellular molecules that modulate PFM/SET expression and activity. As used herein, the term “PFM/SET modulatory compound” refers to a molecule that alters PFM/SET expression or activity. A PFM/SET modulatory compound can increase or decrease PFM/SET expression or activity. Such compounds can be used in ex vivo and in vivo therapeutic applications, as described further below, to promote or inhibit cell proliferation.

As disclosed herein, the PR and SET domains of the PFM/SET polypeptides of the invention have methyltransferase enzymatic activity and function as specific protein binding domains. Through the methyltransferase activity and specific binding to particular cellular proteins, the intact PR/SET domain contributes to the function of PFM/SET polypeptide as a suppressor of cell growth. Therefore, a PFM/SET modulatory compound can alter the activity of a PFM/SET polypeptide by modulating the enzymatic activity or the molecular interactions of a PFM/SET with another molecule. A PFM/SET modulatory compound can also alter the amount of a PFM/SET polypeptide expressed in a cell, for example, by increasing or decreasing the cellular expression level or stability of a PFM/SET polypeptide.

A PFM/SET modulatory compound can be identified by contacting a PFM/SET-expressing cell with a candidate compound under conditions that allow PFM/SET expression or activity. Expression of a PFM/SET refers to the generation of a PFM/SET mRNA or polypeptide. Therefore, an amount of PFM/SET expression can be represented by an amount of messenger RNA (mRNA) or an amount of polypeptide corresponding to a PFM/SET mRNA. An amount of PFM/SET mRNA expressed in a particular cell is generally determined by the transcriptional activity of the gene encoding the mRNA as well as the stability of the mRNA. The amount of polypeptide expressed in a particular cell is generally determined by the stability of the polypeptide and susceptibility to proteolysis of the polypeptide. An increase in PFM/SET can result, for example, from an increase in the amount of PFM/SET mRNA resulting from increased transcription of a PFM/SET gene, increased stability of PFM/SET mRNA and reduced degradation of PFM/SET mRNA. An alteration in the amount of PFM/SET polypeptide can result, for example, from increased or decreased stability and increased or decreased proteolysis of a PFM/SET polypeptide. An increase in PFM/SET activity can result, for example, from an increase in the amount of PFM/SET polypeptide or alteration in PFM/SET structure or conformation that leads to increased activity, including a modification of a PFM/SET polypeptide.

A PFM/SET modulatory compound can act to modulate PFM/SET activity by increasing or decreasing the amount of PFM/SET polypeptide in a cell, for example, by stimulating increased PFM/SET mRNA expression. PFM/SET mRNA expression can be modulated, for example, by inducing or derepression the transcription of a PFM/SET gene and by regulating the expression of a cellular protein that acts as a transcription factor to regulate gene expression. A compound can act to modulate the amount of PFM/SET activity by increasing or decreasing the stability of a PFM/SET mRNA or polypeptide, for example, by increasing or decreasing a cellular degradation activity, such as a protease activity. Molecules that mediate the regulation of PFM/SET expression, such as receptors and corresponding signal transduction molecules, can also be targets of compounds that increase the expression of PFM/SET in a cell. For example, a signal transduction pathway that stimulates the expression of PFM/SET can be modulated to increase or decrease the level of PFM/SET expression, for example, by increasing or decreasing the rate of PFM/SET synthesis or the length of time that PFM/SET gene expression remains active.

A compound can directly increase or decrease PFM/SET activity, for example, by binding to the enzyme and modulating catalytic activity, such as by inducing a conformational change in the PFM/SET polypeptide. A compound that directly increases or decreases the activity of a PFM/SET polypeptide can be identified by contacting a candidate compound with a PFM/SET polypeptide or functional fragment thereof, contained in a cell, cell fraction, or lysate thereof, and can be an isolated PFM/SET polypeptide. A compound that modulates the interaction of a PFM/SET polypeptide with a binding partner can also be identified by contacting a candidate compound with a PFM/SET polypeptide or functional fragment thereof in the presence of a binding partner. Methods for identifying PFM/SET binding partners are well known to those skilled in the art and are described below.

A compound that binds to and modulates the activity of a PFM/SET polypeptide can be identified using a variety of binding assay formats. A binding assay can use a detectably labeled candidate compound and an unlabeled PFM/SET (and optionally an unlabeled binding partner). Alternatively, a binding assay can use an unlabeled candidate compound or binding partner and a labeled PFM/SET. Other appropriate combinations of labeled and unlabeled molecules can be determined by the skilled person depending on the assay format.

A variety of low- and high-throughput assays known in the art are suitable for detecting specific binding interactions between a PFM/SET nucleic acid molecule or polypeptide and a candidate PFM/SET modulatory compound. These assays include both solution-based methods and solid phase methods (e.g. molecules bound to plates, chips, affinity columns and the like). Binding assays are amenable to either manual or high-throughput automated screening of compounds.

Both direct and competitive binding assays can be performed, including, for example, scintillation proximity assay (SPA) (Alouani, Methods Mol. Biol. 138:135-41 (2000)), UV or chemical cross-linking (Fancy, Curr. Opin. Chem. Biol. 4:28-33 (2000)), competition binding assays (Yamamura et al., Methods in Neurotransmitter Receptor Analysis, Raven Press, New York, 1990), biomolecular interaction analysis (BIA) (Weinberger et al., Pharmacogenomics 1:395-416 (2000)), mass spectrometry (MS) (McLafferty et al., Science 284:1289-1290 (1999) and Degterev, et al., Nature Cell Biology 3:173-182 (2001)), nuclear magnetic resonance (NMR) (Shuker et al., Science 274:1531-1534 (1996), Hajduk et al., J. Med. Chem. 42:2315-2317 (1999), and Chen and Shapiro, Anal. Chem. 71:669A-675A (1999)), and fluorescence polarization assays (FPA) (Degterev et al., supra, 2001) which are incorporated herein by reference. Other assays for detecting specific binding interactions include, for example ELISA assays, FACs analysis, and affinity separation methods, which as described, for example, in Harlow and Lane, Eds. Antibodies: A Laboratory Manual, Cold Spring Harbor Laboratory (1988).

Assays to identify compounds that modulate PFM/SET gene expression can involve first transducing cells with a PFM or SET promoter-reporter nucleic acid construct such that a change in expression of a protein such as β-lactamase, luciferase, green fluorescent protein or β-galactosidase will be detected in response to contacting the cell with a PFM/SET modulatory compound that upregulates or downregulates expression of PFM or SET. Assays and reporter systems useful for monitoring gene expression are well known in the art. Other assays to identify compounds that modulate PFM/SET gene expression include assays that measure levels of PFM or SET transcripts, such as Northern blots, RNase protection assays, and RT-PCR.

Assays to identify compounds that modulate PFM/SET polypeptide expression can involve detecting a change in PFM or SET polypeptide abundance in response to contacting the cell with a PFM or SET modulatory compound. Assays for detecting changes in polypeptide expression include, for example, immunoassays with specific PFM or SET antibodies, such as immunofluorescence, immunohistochemistry and immunoprecipitation assays.

Appropriate assays to determine whether a PFM/SET modulatory compound affects PFM or SET activity so as to inhibit or promote cell proliferation, can be determined by those skilled in the art. The skilled artisan appreciates that molecular pathways involved in cell proliferation are generally well conserved among eukaryotic organisms. Therefore, a proliferation assay can be performed in any eukaryotic cell type in which altered proliferation can be detected including, for example, primary mammalian cells, normal and transformed mammalian cell lines, yeast, insect cells and amphibian cells.

A molecule that modulates cell proliferation can, for example, cause cell cycle arrest at a particular stage of mitosis or meiosis, induce or prevent apoptosis, or promote progression through the cell cycle when normal cells would arrest. Such qualitative changes in the cell cycle can be determined by methods known in the art, and which depend on the cell type used in the assay. A molecule that modulates cell proliferation can also, for example, cause faster or slower progression through the cell cycle, resulting in an increased or decreased number of cells in the population after a given period of time. Those skilled in the art can choose an appropriate assay to determine whether, and by what mechanism, a molecule of the invention affects cell proliferation.

A molecule that modulates cell proliferation can also restore more normal proliferative characteristics to a neoplastic cell. Such a molecule can advantageously be used in therapeutic applications to prevent or treat cancer. To determine whether a molecule of the invention restores more normal proliferative characteristics on a neoplastic cell, an assay can be performed in a mammalian cell that exhibits neoplastic proliferative characteristics, such as soft agar colony formation, overgrowth of a cell monolayer, proliferation in low serum, abnormally rapid proliferation, or tumor formation in an animal. Such cells are known in the art and include both tumor cell lines and primary tumor cells. A molecule of the invention can be introduced or expressed in such a cell, and a determination can be made whether the molecule restores more normal proliferative characteristics to the cell, such as slower growth in culture, fewer foci, fewer soft agar colonies, or a reduction in tumor size, as compared to the parental cell.

The methods of identifying a PFM/SET modulatory compound can involve measuring changes in gene expression by determining the amount of mRNA or polypeptide present in a sample. Methods for measuring both mRNA and polypeptide quantity are well known in the art. Methods for measuring mRNA typically involve detecting nucleic acid molecules by specific hybridization with a complementary probe in solution or solid phase formats. Such methods include northern blots, polymerase chain reaction after reverse transcription of RNA (RT-PCR), and nuclease protection. Measurement of a response of a pathway component can be performed using large scale gene expression methods. For methods of the invention that involve identifying a candidate atherosclerosis drug target molecule, described below, large scale gene expression methods can be advantageously used to measure a large population of expressed genes in an organ, tissue or cell. Examples of methods well known in the art applicable to measuring a change in expression of a population of genes include cDNA sequencing, clone hybridization, differential display, subtractive hybridization, cDNA fragment fingerprinting, serial analysis of gene expression (SAGE), and DNA microarrays.

A variety of methods well known in the art can be used to determine protein levels either directly or indirectly. Such methods include immunochemical methods, such as western blotting, ELISA, immunoprecipitation, and RIA, gel electrophoresis methods including one and two-dimensional gels, methods based on protein or peptide chromatographic separation, methods that use protein-fusion reporter constructs and calorimetric readouts, methods based on characterization of actively translated polysomal mRNA, and mass spectrometric detection.

The methods of the invention for identifying a PFM/SET modulatory compound can involve determining an activity of PFM/SET. Exemplary activities include, for example, transcriptional activity and methyltransferase activity (see, for example, Huang et al., J. Biol. Chem. 273:15933-15939 (1998). Suitable assays for identifying compounds that modulate PFM/SET transcriptional activation, repression and coactivation function can be determined by the skilled person. Such assays are generally based on co-expression of PFM/SET and an appropriate promoter-linked reporter gene in a cell, under conditions where a certain amount of transcription occurs, contacting the cell with the candidate compound, and determining whether there is a change (i.e. either an increase or decrease) in transcriptional activity. Transcription based assays are well known in the art, and readily amenable to high-throughput screening assays.

Methyltransferase activity assays are also well known in the art, and generally involve measuring the amount of transfer of a labeled methyl group from a donor molecule (e.g. radiolabeled S-adenosyl- methionine) to a protein, and determining any change in this activity in response to a candidate compound.

Methyltransferase activity assays are described, for example, in Chen et al., Science 284:2174-2177 (1999).

PF, SET, PRAZ and PKZL domains of a PFM/SET polypeptide can bind to cellular proteins and thereby regulate a PFM/SET biological activity. Therefore, an isolated PFM/SET polypeptide, including a functional fragment of a PFM/SET polypeptide that contains a PR, SET, PRAZ or PKZL domain, can be used in assays to identify compounds that modulate the interaction of a PFM/SET polypeptide with another cellular molecule.

Various binding assays to identify cellular proteins that interact with protein binding domains are known in the art and include, for example, yeast two-hybrid screening assays (see, for example, Luban et al., Curr. Opin. Biotechnol. 6:59-64 (1995)) and affinity column chromatography methods using cellular extracts. By synthesizing or expressing polypeptide fragments containing various PFM or SET sequences or deletions, the PFM or SET binding interface can be readily identified.

As further disclosed herein, the zinc finger (ZF) domain of the PFM polypeptides of the invention functions as a specific DNA binding domain. By specifically binding particular DNA sequences, the ZF domain contributes to the function of PFM/SET polypeptides as a suppressor of cell growth. Thus, an isolated PFM/SET polypeptide of the invention containing a ZF domain, or one or more ZF motifs therefrom, can be used, for example, in binding assays to identify cellular DNA sequences that normally bind PFM. Such cellular DNA sequences are likely to be regulatory sequences for genes which themselves have positive or negative growth modulating activity, and which are appropriate targets for therapeutic intervention to prevent or treat proliferative disorders. Furthermore, oligonucleotides or analogs corresponding the PFM binding DNA sequences, can be administered as therapeutic compounds to specifically interfere with PFM function. Additionally, the ZF domain, or one or more ZF motifs therefrom, can be administered as therapeutic compounds to specifically interfere with PFM function.

Various assays to identify DNA sequences that bind DNA binding domains are known in the art and include, for example, Cyclic Amplification and Selection of Targets (CASTing), as described by Wright et al., Mol. Cell. Biol. 11:4104-4110 (1991), and the Multiplex Selection Technique (MuST), as described by Nallur et al., Proc. Natl. Acad. Sci. USA 93:1184-1189 (1996).

As understood by those of skill in the art, assay methods for identifying compounds that increase PFM/SET activity generally require comparison to a control. One type of a “control” is a cell or isolated PFM/SET polypeptide preparation that is treated substantially the same as the test cell exposed to a candidate compound, except that a control is not exposed to a compound. A control cell or isolated PFM/SET polypeptide can be treated with a carrier solution or solvent in which a candidate compound is dissolved or contained, such as an aqueous or organic solution, if desired.

Given the teachings and guidance provided herein, the choice of measuring MRNA or polypeptide amount or polypeptide activity will be that of the user. Considerations such as the sample type, availability and amount will also influence selection of a particular assay format. For example, if a small amount of sample is available, formats which are more sensitive are suitable. Alternatively, if the user is analyzing numerous different samples simultaneously, a multisample format is suitable. Those skilled in the art will know, or can determine, which format is useful for a particular application and which methods or modifications of methods known in the art are compatible with a particular assay format.

A PFM/SET modulatory compound can be a naturally occurring macromolecule, such as a peptide, nucleic acid, carbohydrate, lipid, or any combination thereof. A PFM/SET modulatory compound also can be a partially or completely synthetic derivative, analog or mimetic of such a macromolecule, or a small organic or inorganic molecule prepared partly or completely by combinatorial chemistry methods.

Methods for producing pluralities of compounds to use in screening for PFM/SET modulatory compounds, including chemical or biological molecules such as simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources.

Therapeutic applications

As disclosed herein, PFM/SET nucleic acid molecules encoding PFM or SET polypeptides with intact PR/SET domains inhibit cell growth. In contrast, PFM/SET nucleic acid molecules encoding PFM or SET polypeptides with partial PR/SET domains, or lacking PR/SET domains, promote cell growth. Thus, by selectively manipulating the expression or activity of either the PR/SET+ or PR/SET− forms of the PFM/SET molecules of the invention, or both, it is readily apparent that cell growth can be modulated in either a positive or negative manner, as desired.

Accordingly, the invention provides PFM/SET molecules and therapeutic methods that can be used to inhibit the growth of cells in culture, or in a subject. Advantageously, the molecules and therapeutic methods can be used to treat proliferative disorders in a subject. As used herein, the term “proliferative disorder” refers to a condition in which unwanted cell proliferation of one or more subset of cells in a mammal, such as a human, occurs, resulting in harm (e.g., discomfort or decreased life expectancy) to the mammal. Cell proliferative disorders include diseases such as cancer, in which the cells are neoplastically transformed, but also include diseases resulting from overgrowth of normal cells. For example, cell proliferative disorders include diseases associated with the overgrowth of connective tissues, such as various fibrotic diseases, including scleroderma, arthritis, alcoholic liver cirrhosis, keloid, and hypertropic scarring; vascular proliferative disorders, such as atherosclerosis; and benign tumors.

The invention also provides PFM/SET molecules and therapeutic methods that can be used to enhance proliferation of normal cells. For some therapeutic applications, it may be useful to increase the proliferation of normal cells, without rendering the cells cancerous. In particular, in diseases of cell degeneration, such as Duchenne's muscular dystrophy, insulin-dependent diabetes mellitus, Parkinson's disease, Huntington's disease, Alzheimer's disease, paralysis, cerebellar atrophy, and the like, it may be useful to remove some of the remaining normal cells from the affected tissue of the individual, and culture the cells in large numbers ex vivo for reimplantation into the patient. Additionally, in applications such as wound healing and skin grafts, it is often desirable to increase the proliferation of normal cells.

In one embodiment, the invention provides a method for modulating cell growth, by introducing a vector containing an isolated PFM6, PFM7, PFM8, PFM9, PFM10, PFM11, PFM12, PFM13, PFM14 or SET07 nucleic acid molecule of the invention, operatively linked to a promoter of RNA expression, into a host cell, and expressing the encoded PFM/SET polypeptide in an amount effective to modulate growth of the cell. For applications in which inhibition of cell growth is desirable, expression vectors containing isolated full-length PFM/SET nucleic acid molecules, or modifications of full-length PFM/SET nucleic acid molecules that retain the growth inhibitory activity of PFM/SET, can be introduced into cells under conditions in which the PFM/SET polypeptide is expressed in an effective amount to inhibit cell proliferation. For applications in which promotion of cell growth is desirable, expression vectors which contain portions of the PFM/SET nucleic acid molecules that compete with PFM/SET for substrates or effectors, such as the PR domain or zinc finger domain, or both, can be introduced into cells under conditions in which the PFM/SET polypeptide is expressed in an effective amount to promote cell proliferation.

Various gene therapy strategies are well known to those skilled in the art, and are reviewed, for example in Roth et al., Oncoloqy 13(10 Suppl 5):148-54, (1999). Useful mammalian expression vectors for such therapeutic applications, and methods of introducing such vectors into mammalian cells either ex vivo or in vivo, for expression of the encoded polypeptide, are well known in the art. Viruses are specialized infectious agents that can elude host defense mechanisms and can infect and propagate in specific cell types. Viral based systems provide the advantage of being able to introduce relatively high levels of the heterologous nucleic acid into a variety of cells. Suitable viral vectors for introducing an invention PFM/SET nucleic acid into mammalian cells (e.g., vascular tissue segments) are well known in the art. These viral vectors include, for example, Herpes simplex virus vectors (Latchman, Histology and Histopathology, 15:1253-1259 (2000)), Vaccinia virus vectors, Cytomegalovirus vectors, Moloney murine leukemia virus vectors, adenovirus vectors, adeno-associated virus vectors, retrovirus vectors, lentiviral vectors and the like.

In particular, the specificity of viral vectors for particular cell types can be utilized to target predetermined cell types. Thus, the selection of a viral vector will depend, in part, on the cell type to be targeted. For example, if a proliferative disease is to be treated by increasing the level of a PFM/SET polypeptide in a particular tissue affected by the disease, then a viral vector that targets the particular tissue can be used. For example, if a disease or pathological condition of the hematopoietic system is to be treated, then a viral vector that is specific for a particular blood cell or its precursor cell can be used. A vector based on a human immunodeficiency virus is an example of such a viral vector. In addition, a viral vector or other vector can be constructed to express a PFM/SET nucleic acid in a tissue specific manner by incorporating a tissue-specific promoter or enhancer into the vector.

Non-viral synthetic vectors and hybrid vectors are also useful for introducing a PFM/SET nucleic acid molecule of the invention into a cell. Cell-based delivery methods involving ex vivo genetic manipulation of cells are also useful for delivering a PFM/SET nucleic acid molecule into a cell of an individual.

For gene therapy, a vector containing a PFM/SET nucleic acid or an antisense nucleotide sequence can be administered to a subject by various methods. For example, if viral vectors are used, administration can take advantage of the target specificity of the vectors. In such cases, there in no need to administer the vector locally at the diseased site. However, local administration can be a particularly effective method of administering a PFM/SET nucleic acid. In addition, administration can be via intravenous or subcutaneous injection into the subject. Following injection, the viral vectors will circulate until they recognize host cells with the appropriate target specificity for infection. Injection of viral vectors into the spinal fluid also can be an effective mode of administration, for example, in treating a neurodegenerative disease.

Receptor-mediated DNA delivery approaches also can be used to deliver a PFM/SET nucleic acid molecule into cells in a tissue-specific manner using a tissue-specific ligand or an antibody that is non-covalently complexed with the nucleic acid molecule via a bridging molecule. Direct injection of a naked or a nucleic acid molecule encapsulated, for example, in cationic liposomes also can be used for stable gene transfer into non-dividing or dividing cells in vivo. In addition, a PFM/SET nucleic acid molecule can be transferred into a variety of tissues using the particle bombardment method. Such nucleic acid molecules can be linked to the appropriate nucleotide sequences required for transcription and translation.

A particularly useful mode of administration of a PFM/SET nucleic acid is by direct inoculation locally at the site of the disease or pathological condition. Local administration can be advantageous because there is no dilution effect and, therefore, the likelihood that a majority of the targeted cells will be contacted with the nucleic acid molecule is increased. Thus, local inoculation can alleviate the targeting requirement necessary with other forms of administration and, if desired, a vector that infects all cell types in the inoculated area can be used. If expression is desired in only a specific subset of cells within the inoculated area, then a promoter, an enhancer or other expression element specific for the desired subset of cells can be linked to the nucleic acid molecule. Vectors containing such nucleic acid molecules and regulatory elements can be viral vectors, viral genomes, plasmids, phagemids and the like. Transfection vehicles such as liposomes also can be used to introduce a non-viral vector into recipient cells. Such vehicles are well known in the art.

In another embodiment, the invention provides a method for modulating cell growth by introducing into a cell an effective amount of an antisense oligonucleotide or a ribozyme that inhibits expression of PFM/SET, thereby modulating growth of the cell. Methods for modulating gene expression using antisense oligonucleotides and ribozymes are also well known in the art. Thus, an antisense molecule or ribozyme that selectively inhibits expression of the PR/SET−, growth promoting form of PFM/SET, can be used to inhibit cell proliferation. In contrast, an antisense molecule or ribozyme that selectively inhibits expression of the PR+, growth suppressing form of PFM/SET, can be used to promote cell proliferation.

Antisense oligonucleotides that inhibit PFM/SET gene expression generally are at least about 17 nucleotides in length, and often include sequences found within the first 30 nucleotides of the transcript being targeted. The preparation and use of antisense oligonucleotides are well known in the art and described in detail, for example, in Cohen (ed), Oligonucleotides: Antisense Inhibitors of Gene Expression, CRC Press Inc., Boca Raton (1989). Likewise, methods of preparing and using hairpin and hammerhead ribozymes for the selective inhibition of gene expression are known in the art and are described, for example, in Poeschla et al., Curr. Olin. Oncol. 6:601-606 (1994).

In a further embodiment, the invention provides a method for modulating cell growth by contacting the cell with an effective amount of a PFM/SET modulatory compound. Methods of identifying PFM/SET modulatory compounds have been described above.

In yet another embodiment, the invention provides a method for modulating cell growth by administering antibodies that specifically bind a PFM or SET polypeptide. For example, antibodies that selectively detect a growth promoting structural variant of PFM or SET, such as the PR− form of PFM polypeptide, can be administered to selectively target cells that express this variant. If desired, such antibodies can be administered in conjunction with a cytotoxic or cytostatic moiety, such as a radioisotope or toxin, in order to neutralize or kill cells expressing the desired structural variant.

The PFM/SET therapeutic molecules of the invention described herein, including expression vectors, antisense oligonucleotides and ribozymes, PFM/SET modulatory compounds, and antibodies, can optionally be formulated together with a pharmaceutically acceptable carrier for delivery to a cultured cell or to a subject. Suitable pharmaceutically acceptable carriers are well known in the art and include, for example, aqueous or organic solvents such as physiologically buffered saline, glycols, glycerol, oils or injectable organic esters. A pharmaceutically acceptable carrier can also contain a physiologically acceptable compound that acts, for example, to stabilize or increase the solubility of a pharmaceutical composition. Such a physiologically acceptable compound can be, for example, a carbohydrate, such as glucose, sucrose or dextrans; an antioxidant, such as ascorbic acid or glutathione; a chelating agent; a low molecular weight protein; or another stabilizer or excipient. Pharmaceutically acceptable carriers, including solvents, stabilizers, solubilizers and preservatives, are well known to those skilled in the art.

Those skilled in the art can formulate the therapeutic molecules to ensure proper distribution in vivo. For example, the blood-brain barrier (BBB) excludes many highly hydrophilic compounds. To ensure that the therapeutic molecules of the invention cross the BBB, if desired, they can be formulated, for example, in liposomes, or chemically derivatized. Methods of ensuring appropriate distribution in vivo can also be provided by rechargeable or biodegradable devices, particularly where gradients of concentrations of drug in a tissue are desired. Various slow release polymeric devices are known in the art for the controlled delivery of drugs, and include both biodegradable and non- degradable polymers and hydrogels. Those skilled in the art understand that the choice of the pharmaceutical formulation and the appropriate preparation of the composition will depend on the intended use and mode of administration.

The therapeutic molecules of the invention, including expression vectors, antisense oligonucleotides and ribozymes, PFM/SET modulatory compounds and antibodies, can be administered to a subject by any effective route. Suitable routes for delivering the therapeutic molecules of the invention include topically, intraocularly, intradermally, parenterally, orally, intranasally, intravenously, intramuscularly, intraspinally, intracerebrally and subcutaneously. In a preferred embodiment, the therapeutic PFM or SET molecules are directly injected into a solid tumor, tumor-containing organ or tumor containing body cavity, in a effective amount to inhibit proliferation of the tumor cells. Alternatively, the therapeutic PFM or SET molecules of the invention can be administered systemically into the blood or lymphatic circulation to reach cells in the circulatory system or in any organ or tissue.

An effective dose of a therapeutic molecule of the invention can be determined, for example, by extrapolation from the concentration required for binding an isolated PFM/SET nucleic acid or polypeptide in binding and functional assays described herein; from the dose required to modulate PFM or SET nucleic acid or polypeptide expression in the expression assays described herein; or from the dose required to modulate cell proliferation in the proliferation assays described herein.

An effective dose of a molecule of the invention for the treatment of proliferative disorders can also be determined from appropriate animal models, such as xenografts of human tumors in rats or mice. Human cancer cells can be introduced into an animal by a number of routes, including subcutaneously, intraveneously and intraperitoneally. Following establishment of a tumor, the animals can be treated with different doses of a molecule of the invention, and tumor mass or volume can be determined. An effective dose for treating cancer is a dose that results in either partial or complete regression of the tumor, reduction in metastasis, reduced discomfort, or prolonged life span.

The appropriate dose for treatment of a human subject with a therapeutic molecule of the invention can be determined by those skilled in the art, and is dependent on the nature and bioactivity of the particular compound, the desired route of administration, the gender, age and health of the individual, the number of doses and duration of treatment, and the particular condition being treated.

Diagnostic applications

The PFM/SET nucleic acids and polypeptides disclosed herein exist in different forms, depending on the splice variant expressed. PFM nucleic acids and polypeptides that express or contain an intact PR domain (PR+) are associated with regulated, or normal, cell proliferation. A decrease in the total amount, or relative amount, of the PR+ form of a PFM, or an increase in the total amount, or relative amount, of the PR− form of a PFM, is associated with unregulated, or pathological, cell proliferation. Therefore, determining the total or relative abundance of the PR+and PR− forms of PFM, or identifying alterations in the expression or structure of PFM nucleic acid molecules or polypeptides, can be used to distinguish between normal and pathologically proliferative cells in a sample. Similarly, detection of the presence or absence of a SET domain in a PFM/SET nucleic acid or polypeptide can be used to distinguish between normal and pathologically proliferative cells in a sample.

The invention thus provides methods of detecting PFM/SET nucleic acids and polypeptides in a sample. As used herein, the term “sample” is intended to mean any biological fluid, cell, tissue, organ or portion thereof, that includes or potentially includes PFM or SET nucleic acids or polypeptides. The term includes samples present in an individual as well as samples obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid or protein preparation. A sample can be prepared by methods known in the art suitable for the particular format of the detection method employed.

The detection methods of the invention can advantageously be used, for example, to identify pathologically proliferative cells, such as neoplastic cells, in a sample. As used herein, the term “neoplastic cell” is intended to mean a cell that exhibits histological or proliferative features of a malignant or premalignant cell. For example, by histological methods, a neoplastic cell can be observed to invade into surrounding normal tissue, have an increased mitotic index, an increased nuclear to cytoplasmic ratio, altered deposition of extracellular matrix, or a less differentiated phenotype. A neoplastic cell can also exhibit unregulated proliferation, such as anchorage independent cell growth, proliferation in reduced-serum medium, loss of contact inhibition, or rapid proliferation compared to normal cells. The diagnostic methods described herein are applicable to the identification of any type of neoplastic cell, such as neoplastic cells present in solid tumors (carcinomas and sarcomas) such as breast, colorectal, gynecological, lung, prostate, bladder, renal, liver, urethral, endocrinal, melanoma, basal cell, central nervous system, lymphoma, stomach, esophageal, squamous cell cancers, as well as all forms of leukemias, and metastases therefrom.

The diagnostic methods described herein can also be adapted for use as prognostic assays. Such an application takes advantage of the observation that alterations in expression or structure of different tumor suppressor molecules take place at characteristic stages in the progression of a proliferative disease or of a tumor. Knowledge of the stage of the tumor allows the clinician to select the most appropriate treatment for the tumor and to predict the likelihood of success of that treatment.

The diagnostic methods described herein can also be used to identify individuals at increased risk of developing a proliferative disease, such as cancer, due to hereditary mutations in a PFM or SET.

The invention thus provides methods for detecting PFM/SET nucleic acid in a sample. In one embodiment, the method consists of contacting the sample with an isolated PFM6, PFM7, PFM8, PFM9, PFM10, PFM11, PFM12, PFM13, PFM14 or SET07 nucleic acid molecule, under conditions that allow specific hybridization to PFM/SET nucleic acid, and detecting specific hybridization.

In another embodiment, the method consists of contacting the sample with a PFM/SET primer pair, under conditions that allow amplification of PFM/SET nucleic acid, and detecting amplified PFM/SET nucleic acid.

The methods of detecting PFM/SET nucleic acid in a sample can be either qualitative or quantitative, as desired. For example, the presence, abundance, integrity or structure of a PFM/SET, or of particular splice variants thereof, can be determined, as desired, depending on the assay format and the probe or primer pair chosen.

Useful assays for detecting PFM/SET nucleic acid based on specific hybridization with an isolated PFM/SET nucleic acid molecule are well known in the art and include, for example, in situ hybridization, which can be used to detect altered chromosomal location of the nucleic acid molecule, altered gene copy number, and RNA abundance, depending on the assay format used. Other hybridization assays include, for example, Northern blots and RNase protection assays, which can be used to determine the abundance and integrity of different RNA splice variants, and Southern blots, which can be used to determine the copy number and integrity of DNA. A PFM/SET hybridization probe can be labeled with any suitable detectable moiety, such as a radioisotope, fluorochrome, chemiluminescent marker, biotin, or other detectable moiety known in the art that is detectable by analytical methods.

Useful assays for detecting PFM/SET nucleic acid in a sample based on amplifying PFM/SET nucleic acid with a PFM/SET primer pair are also well known in the art, and include, for example, qualitative or quantitative polymerase chain reaction (PCR); reverse-transcription PCR (RT-PCR); SSCP analysis, which can readily identify a single point mutation in DNA, such as in a PCR or RT-PCR product; and coupled PCR, transcription and translation assays, such as the Protein Truncation Test, in which a mutation in DNA is determined by an altered protein product on an electrophoresis gel. Additionally, the amplified PFM/SET nucleic acid can be sequenced to detect mutations and mutational hot-spots, and specific assays for large-scale screening of samples to identify such mutations can be developed.

Such assays are also applicable to the qualitative or quantitative detection of SET nucleic acid in a sample. Therefore, the invention thus provides methods for detecting PFM/SET nucleic acid in a sample.

The invention also provides methods for detecting PFM/SET polypeptide in a sample, by contacting the sample with an agent specific for PFM/SET, under conditions that allow specific binding of the agent to PFM/SET polypeptide, and detecting the specifically bound agent. As used herein the term “agent specific for PFM/SET” refers to a molecule that specifically binds PFM/SET polypeptides. An example of a molecule that specifically binds PFM or SET is a PFM or SET antibody, respectively, or antigen binding fragment thereof. Additionally, the PFM/SET binding and modulatory compounds identified in the affinity screening methods described above are also suitable agents that can be used in methods of detecting PFM or SET polypeptides.

Assays for detecting PFM/SET polypeptides include, for example, immunohistochemistry, immunofluorescence, ELISA assays, radioimmunoassay (RIA), FACS analysis, immunoprecipitation, and immunoblot analysis, using antibodies or antigen binding fragments specific for PFM or SET. Various immunoassays are well known in the art, and can be readily modified by those skilled in the art in cases in which the agent is a PFM or SET binding molecule other than an antibody. If desired, the agent or antibody can be rendered detectable by incorporation of, or by conjugation to, a detectable moiety, or binding to a secondary molecule that is itself detectably labeled.

In the detection methods of the invention, the nucleic acid probes or primers, and polypeptide binding agents, can advantageously be directed against the PR domain of PFM, or the A, B or C boxes thereof. Therefore, these assays can be used to distinguish between PR+growth-inhibiting, and PR−, growth-promoting, forms of PFM in a sample.

The following examples are intended to illustrate but not limit the present invention.

The involvement of PFM/SET in cell differentiation and cancer, and the chromosomal locations of PFM/SET genes, as shown in the Examples below, are consistent with a role for PFM/SET in human diseases including cancer.

### EXAMPLE I

### Identification and Characterization of PFM6

This example shows identification of the gene encoding the PR−domain containing polypeptide designated PFM6, and characterization of its structure and chromosomal location.

Human genome sequence databases were screened using the PR domain of RIZ1, BLIMP1 and MDS1-EVI1 as probes or queries. This led to the identification of an unfinished human chromosome 5 genomic sequence (AC010432) encoding a PR domain. Sequence analysis revealed an open reading frame encoding 683 amino acids. The predicted amino acid sequence contains a PR domain, a PKZL domain, and 14 zinc finger domains. This gene was designated PFM6 for PR family member 6.

The PKZL domain stands for “PR and KRAB zinc-finger protein linked” domain since the 100 residue domain exhibits 34% identity to the N-terminal region of the KRAB-domain containing zinc finger protein 133, ZNF133.

The structural features of PFM6 protein suggest a role for the protein as a DNA binding transcription factor. The PR domain of PFM6 is more related to PFM4 (GenBank accession number XM_006873) than to other PR genes.

Using PFM6 human cDNA as a query sequence, an STS (sequence tagged site) sequence, STS294 was identified. This STS marker maps to 5p14. Rearrangements or gains of chromosome 5p are commonly found in ovarian and breast cancers (Sonoda et al., (1997) , supra)

### EXAMPLE II

### Identification and Characterization of PFM7

This example shows identification of the gene encoding the PR-domain containing polypeptide designated PFM7, and characterization of its structure and chromosomal location.

cDNA libraries and human genomic sequence libraries were screening using the PR domain of RIZ1, BLIMP1 and MDS1-EVI1 as probes or queries. This led to the identification of a partial cDNA sequence encoding a protein identified as KIAA1231 (GenBank Accession number AB33057) and a genomic clone on 11q25 (AP000686) that encode a PR domain. Sequence analysis revealed an open reading frame encoding 1061 amino acids. The predicted amino acid sequence contains a PRAZ motif, a PR domain, an acidic motif, and 10 zinc finger motifs. The gene is widely expressed in adult human tissues. This gene was designated PFM7.

Using PFM7 human cDNA as a query sequence, a genomic clone (AP000686), which maps to 11q-25, was identified. The chromosomal locus 11q25 is frequently deleted in breast, ovary, colon and oral cancer (Connolly et al., supra; Koreth et al., Oncogene 14:431-7 (1997); Launonen et al., supra; Uzawa et al., Intl. J. Cancer 67:510-4 (1996)). It has also been demonstrated that llq25 YAC clones can suppress tumor growth in vivo (Koreth et al., Oncogene 18:1157-64 (1999)).

### EXAMPLE III

### Identification and Characterization of PFM8

This example shows identification of the gene encoding the PR−domain containing polypeptide designated PFM8, and characterization of its structure and chromosomal location.

Human genome sequence databases were screened using the PR domain of RIZ1, BLIMP1 and MDS1-EVI1 as probes or queries. This led to the identification of an unfinished human chromosome genomic sequence (AC013602) encoding a PR domain. The full length cDNA was obtained by rapid amplification of cDNA ends (RACE) using a Marathon Race Kit (CLONETECH Laboratories, Inc., Palo Alto, Calif.) with a human brain cDNA library. The primers used for cloning the PFM CDNA sequence of about 0.9 kb were 5′-tgtccctgcacgcccggaagtagatg-3′ (SEQ ID NO:21) and 5′-tgtgctggaacgccagcaggtt-3′ (SEQ ID NO:22). The amplified nucleotide sequence is referenced as SEQ ID NO:25. The obtained cDNA encoded a 504 amino acid sequence predicted to contain a PR domain and a PRAZ domain. This gene was designated PFM8.

STS markers within the genomic clone were identified STS markers having Accession numbers G36915, G37367, G13751, and G52735. The STS markers that map 11p11, a region commonly deleted in breast cancer (Nakata et al., supra).

### EXAMPLE IV

### Characterization of PFM9

This example shows identification of the gene encoding the PR−domain containing polypeptide designated PFM9, and characterization of its structure and chromosomal location.

cDNA libraries and human genomic sequence libraries were screening using the PR domain of RIZ1, BLIMP1 and MDS1-EVI1 as probes or queries. This led to the identification of a an unfinished genomic clone (AC015497) encoding a PR domain. Sequence analysis revealed an open reading frame encoding 364 amino acids. The predicted amino acid sequence contains a PR domain and 3 zinc finger motifs. This gene was designated PFM9.

To map the chromosomal location of PFM9, the Stanford radiation hybrid panel was screened with a pair of PCR primers that amplify PMF9 gene. This mapped PFM9 to chromosome 9q33-34.1. This region is commonly deleted in ovarian, bladder, esophageal, and lung cancers (Devlin et al., supra; Hornigold et al., supra; and Simoneau et al., Oncogene 18:157-63 (1999)).

### EXAMPLE V

### Characterization of PFM10

This example shows identification of the gene encoding the PR−domain containing polypeptide designated PFM10, and characterization of its structure and chromosomal location.

cDNA libraries and human genomic sequence libraries were screening using the PR domain of RIZ1, BLIMP1 and MDS1-EVI1 as probes or queries. This led to the identification of a an unfinished genomic clone on chromosome 6q16.1-21 (AL035087) encoding a PR domain. Sequence analysis revealed an open reading frame encoding 717 amino acids. The predicted amino acid sequence contains a PR motif and 4 zinc finger motifs.

To map the chromosomal location of PFM10, the Stanford radiation hybrid panel was screened with a pair of PCR primers that amplify PMF10 gene. This mapped PFM10 to chromosome 6q16-21. The 6q16-21 region is commonly deleted in B-cell lymphoma, melanoma, and stomach cancers. This region also contains another PR-family member, the candidate tumor suppressor PRDIBF1/BLIMP1 (PRDM1) (Mock et al., supra).

### EXAMPLE VI

### Characterization of PFM11

This example shows identification of the gene encoding the PR−domain containing polypeptide designated PFM11, and characterization of its structure and chromosomal location.

cDNA libraries and human genomic sequence libraries were screening using the PR domain of RIZ1, BLIMP1 and MDS1-EVI1 as probes or queries. This led to the identification of a genomic clone (AC018740) that encodes a PR domain. Sequence analysis revealed an open reading frame encoding 518 amino acids. The predicted amino acid sequence contains a PR domain.

To map the chromosomal location of PFM11, the Stanford radiation hybrid panel was screened with a pair of PCR primers that amplify PMF11 gene. This mapped PFM11to chromosome 8p12-21, a region commonly deleted in breast and prostate cancers (Van Alewijk et al., Genes, Chromosomes & Cancer 24:119-26 (1999); Verma et al., supra; and Vocke et al., supra).

### EXAMPLE VII

### Characterization of PFM12

This example shows identification of the gene encoding the PR−domain containing polypeptide designated PFM12, and characterization of its structure and chromosomal location.

CDNA libraries and human genomic sequence libraries were screening using the PR domain of RIZ1, BLIMP1 and MDS1-EVI1 as probes or queries. This led to the identification of a sequence on chromosome 21 that encodes a PR domain. Sequence analysis revealed an open reading frame encoding 951 amino acids. The predicted amino acid sequence contains a PR domain, PRAZ motif and 8 C2H2 zinc finger domains.

To map the chromosomal location of PFM12, the Stanford radiation hybrid panel was screened with a pair of PCR primers that amplify PMF12. This mapped PFM12 to chromosome 21q22.3, a region deleted in human leukemia (Hoffman et al. Annals of Oncology, 6(8):781-8 (1995), Pederson-Bjergaard et al. Leukemia, 7(12):1975- 86 (1993) and Rubin et al. Blood, 76(12):2594-8 (1990)) and bladder cancer (Babu et al. Cancer Genetics and Cytogenetics 38(1):127-9 (1989)).

### EXAMPLE VIII

### Characterization of PFM13

This example shows identification of the gene encoding the PR−domain containing polypeptide designated PFM13, and characterization of its structure and chromosomal location.

cDNA libraries and human genomic sequence libraries were screening using the PR domain of RIZ1, BLIMP1 and MDS1-EVI1 as probes or queries. An EST clone (AI278689) was initially identified having a partial PR domain. Sequence of the cDNA was obtained by RACE, as described above in relation to PFM8, and by analysis of sequences in the public databases. The primers used for amplifying the PFM13 cDNA were 5′-gggggtagacgccttggttcacg-3′ (SEQ ID NO:23), and 5′-catcgcaggagcacgccacac-3′ (SEQ ID NO:24). The amplified nucleotide sequence is referenced as SEQ ID NO:26. The full length cDNA of 4367 bp is predicted to encode 1257 amino acids. The predicted amino acid sequence contains a PR domain and 10 zinc finger domains. The sequence shares 51% peptide sequence identity with the MDS1-EVI1 cancer gene over the entire length of the protein, and is therefore a paralog of MDS1-EVI1.

By STS content mapping, the PFM13 gene was mapped to chromosome band 1p36.23-33, a region deleted in more than a dozen different types of human cancers. The proven role of MDS1-EVI1 and RIZ1 (also on 1p36) in human cancers (Huang, 1999), suggests a role for PFM13 as one of several tumor suppressors thought to reside on 1p36.

### EXAMPLE IX

### Characterization of PFM14

This example shows identification of the gene encoding the PR-domain containing polypeptide designated PFM14, and characterization of its structure and chromosomal location.

cDNA libraries and human genomic sequence libraries were screening using the PR domain of RIZ1, BLIMP1 and MDS1-EVI1 as probes or queries. An EST clone (BE732157.1) was initially identified having a partial PR domain. The EST clone was used to identify a genomic sequence (AC12054) on chromosome 11, which was then used to predict an open reading frame of 720 amino acids. The predicted amino acid sequence of PFM14 contains one PR domain and 10 zinc finger motifs.

### EXAMPLE X

### Characterization of SET07

This example shows identification of the gene encoding the SET-domain containing polypeptide designated SET07, and characterization of its structure and chromosomal location.

cDNA libraries and human genomic sequence libraries were screening using the SET domain of human HRX as a query. An EST clone (AA085455) was found to contain a SET domain. A full length cDNA containing the SET domain was assembled using sequences in the public databases. A genomic sequence (AC005283) was identified to contain the SET domain gene, termed SET07. An STS identified to correspond to the genomic sequence was used to map SET07 to chromosome 13q11-q13, in proximity to the retinoblastoma locus on 13q14. This region is commonly deleted in many cancers including breast cancers, bladder cancers, lung cancers and osteosarcomas.

### EXAMPLE XI

### PFM/SET Modulation of Cell Proliferation

This Example describes a method for demonstrating the ability of a PFM/SET nucleic acid to modulate cell proliferation.

A human PFM/SET cDNA is inserted into a mammalian expression vector, such as pCMV-Tag4, to generate an expression construct encoding PFM/SET. The encoded PFM/SET polypeptide can be tagged with a detectable epitope, for example, with a myc, GST or FLAG epitope tag, at the C-terminus. Alternatively, the desired epitope can be placed at the N-terminus, or at an internal site, so long as the epitope does not interfere with PFM/SET activity. The PFM/SET expression construct is transfected, either transiently or stably, into cells (for example, 293 or NIH-3T3 cells) using a commercially available reagent, such as Effectene or Superfect transfection reagents (Qiagen, Valencia, Calif.). Expression of the PFM/SET protein from the construct is then confirmed by detecting the epitope or the PFM/SET polypeptide, for example, by immunostaining of cells or immunoblot analysis.

Cell proliferation in cells having or lacking PFM/SET expression is then determined. One well known method for measuring cell proliferation is by measuring BrdU incorporation. BrdU incorporation into DNA is a measure of DNA synthesis, which is required for cell proliferation. The effect of PFM/SET expression on cell proliferation is measured by growing cells for 2 h in BrdU-containing media following transfection, staining the cells with anti-BrdU monoclonal antibody and DAPI (nuclear staining reagent), and counting the number of BrdU positive and negative cells in several different microscopic views.

In this manner, the effect of PFM/SET expression on cell proliferation can be determined. A variety of other cell proliferation assays are well known to those skilled in the art, and can also be used for determining cell proliferation in cells lacking or expressing a PFM/SET polypeptide.

All journal article, reference and patent citations provided above, including referenced sequence accession numbers of nucleotide and amino acid sequences contained in various databases, in parentheses or otherwise, whether previously stated or not, are incorporated herein by reference in their entirety.

Although the invention has been described with reference to the examples provided above, it should be understood that various modifications can be made without departing from the spirit of the invention.

What is claimed is:

1. An isolated nucleic acid molecule encoding a PFM/SET polypeptide, said PFM/SET polypeptide having at least about 80% identity with an amino acid sequence referenced as SEQ ID NO:2, 4, 6, 8, 10, 12, 14, 16, 18 or 20.

2. The isolated nucleic acid molecule of claim 1, encoding a PFM/SET polypeptide having at least about 90% identity with an amino acid sequence referenced as SEQ ID NO:2, 4, 6, 8, 10, 12, 14, 16, 18 or 20.

3. The isolated nucleic acid molecule of claim 1, encoding a PFM/SET polypeptide having at least about 95% identity with an amino acid sequence referenced as SEQ ID NO:2, 4, 6, 8, 10, 12, 14, 16, 18 or 20.

4. The isolated nucleic acid molecule of claim 1, encoding a PFM/SET polypeptide comprising an amino acid sequence referenced as SEQ ID NO:2, 4, 6, 8, 10, 12, 14, 16, 18 or 20.

5. The isolated nucleic acid molecule of claim 1, encoding a PFM/SET polypeptide consisting of an amino acid sequence referenced as SEQ ID NO:2, 4, 6, 8, 10, 12, 14, 16, 18 or 20.

6. The isolated nucleic acid molecule of claim 1, wherein said PFM/SET polypeptide is encoded by a nucleotide sequence comprising a nucleotide sequence selected from the group consisting of SEQ ID NOS:1, 3, 5, 7, 9, 11, 13, 15, 17 and 19.

7. The isolated nucleic acid molecule of claim 6, wherein said PFM/SET polypeptide is encoded by a nucleotide sequence selected from the group consisting of SEQ ID NOS:1, 3, 5, 7, 9, 11, 13, 15, 17 and 19.

8. An isolated nucleic acid molecule, comprising a nucleotide sequence encoding a functional fragment of a PFM/SET polypeptide, said fragment comprising a PR, SET, PRAZ, or PKZL domain of a PFM/SET amino acid sequence selected from the group consisting of SEQ ID NOS:2, 4, 6, 8, 10, 12, 14, 16, 18 and 20.

9. A vector, comprising the isolated nucleic acid molecule of claim 1.

10. The vector of claim 8, wherein said isolated nucleic acid molecule is operatively linked to a promoter of RNA transcription.

11. A host cell, comprising the vector of claim 10.

12. An isolated oligonucleotide, comprising at least 17 contiguous nucleotides of a nucleotide sequence referenced as SEQ ID NO:1,3, 5, 7, 9, 11, 13, 15, 17 or 19, or the complement thereof.

13. A primer pair for detecting a PFM/SET nucleic acid molecule, comprising two isolated oligonucleotides according to claim 12.

14. A method for detecting a PFM/SET nucleic acid molecule in a sample, comprising contacting said sample with the PFM/SET nucleic acid molecule of claim 1 under conditions that allow specific hybridization to PFM/SET nucleic acid, and detecting said specific hybridization.

15. A method for detecting a PFM/SET nucleic acid molecule in a sample, comprising contacting said sample with the primer pair of claim 13 under conditions that allow amplification of PFM/SET nucleic acid, and detecting amplified PFM/SET nucleic acid.

16. A method for modulating cell growth, comprising introducing the vector of claim 10 into a host cell, and expressing encoded PFM/SET polypeptide in an amount effective to modulate growth of said cell.

17. A method for isolating a PFM/SET polypeptide, comprising growing the host cell of claim 11 under conditions appropriate for expression of encoded PFM/SET polypeptide, and isolating said PFM/SET polypeptide.

18. An isolated PFM/SET polypeptide, comprising a PFM/SET amino acid sequence having at least about 80% amino acid identity with an amino acid sequence selected from the group consisting of SEQ ID NO:2, 4, 6, 8, 10, 12, 14, 16, 18 and 20.

19. The isolated PFM/SET polypeptide of claim 18, having at least about 90% amino acid identity with a PFM/SET amino acid sequence selected from the group consisting of SEQ ID NOS:2, 4, 6, 8, 10, 12, 14, 16, 18 and 20.

20. The isolated PFM/SET polypeptide of claim 18, having at least about 95% amino acid identity with a PFM/SET amino acid sequence selected from the group consisting of SEQ ID NOS:2, 4, 6, 8, 10, 12, 14, 16, 18 and 20.

21. An isolated PFM/SET polypeptide, comprising a PFM/SET amino acid sequence selected from the group consisting of SEQ ID NOS:2, 4, 6, 8, 10, 12, 14, 16, 18 and 20.

22. The isolated PFM/SET polypeptide of claim 21, consisting of a PFM/SET amino acid sequence selected from the group consisting of SEQ ID NOS:2, 4, 6, 8, 10, 12, 14, 16, 18 and 20.

32. A functional fragment of a PFM/SET polypeptide, said fragment comprising a PR, SET, PRAZ, or PKZL domain of a PFM/SET amino acid sequence selected from the group consisting of SEQ ID NOS:2, 4, 6, 8, 10, 12, 14, 16, 18 and 20.

33. An isolated immunogenic PFM/SET peptide, comprising at least 8 contiguous amino acids of an amino acid sequence selected from the group consisting of SEQ ID NOS:2, 4, 6, 8, 10, 12, 14, 16, 18 and 20.

34. An antibody, or antigen binding fragment thereof, which specifically binds to a PFM/SET polypeptide comprising an amino acid sequence selected from the group consisting of SEQ ID NOS:2, 4, 6, 8, 10, 12, 14, 16, 18 and 20.

35. A method for detecting PFM/SET polypeptide in a sample, comprising contacting said sample with the antibody of claim 34 under conditions that allow specific binding of said antibody to PFM/SET polypeptide, and detecting specifically bound antibody.
